SEQUENCE LISTING (12 sequences)

SEQ ID NO: 1 - protein, 447 aa, Mus musculus
MIEDNKENKENKDHSSERGRVTLIFSLENEVGGLIKVLKIFQENHVSLLHIESRKSKQRNSEFEIFVDCDISREQLNDIFPLLKSHATVLSVDSPDQLTAKEDVMETVPWFPKKISDLDFCANRVLLYGSELDADHPGFKDNVYRRRRKYFAELAMNYKHGDPIPKIEFTEEEIKTWGTIFRELNKLYPTHACREYLRNLPLLSKYCGYREDNIPQLEDVSNFLKERTGFSIRPVAGYLSPRDFLSGLAFRVFHCTQYVRHSSDPLYTPEPDTCHELLGHVPLLAEPSFAQFSQEIGLASLGASEETVQKLATCYFFTVEFGLCKQDGQLRVFGAGLLSSISELKHALSGHAKVKPFDPKIACKQECLITSFQDVYFVSESFEDAKEKMREFAKTVKRPFGLKYNPYTQSVQVLRDTKSITSAMNELRYDLDVISDALARVTRWPSV

SEQ ID NO: 2 - protein, 488 aa, Mus musculus
MQPAMMMFSSKYWARRGLSLDSAVPEDHQLLGSLTQNKAIKSEDKKSGKEPGKGDTTESSKTAVVFSLKNEVGGLVKALRLFQEKHVNMLHIESRRSRRRSSEVEIFVDCECGKTEFNELIQLLKFQTTIVTLNPPESIWTEEEDLEDVPWFPRKISELDRCSHRVLMYGTELDADHPGFKDNVYRQRRKYFVDVAMGYKYGQPIPRVEYTEEETKTWGVVFRELSKLYPTHACREYLKNLPLLTKYCGYREDNVPQLEDVSMFLKERSGFTVRPVAGYLSPRDFLAGLAYRVFHCTQYVRHGSDPLYTPEPDTCHELLGHVPLLADPKFAQFSQEIGLASLGASDEDVQKLATCYFFTIEFGLCKQEGQLRAYGAGLLSSIGELKHALSDKACVKSFDPKTTCLQECLITTFQDAYFVSDSFEEAKEKMRDFAKSITRPFSVYFNRYTQSIEILKDTRSIENVVQDLRSDLNTVCDALNKMNQYLGI

SEQ ID NO: 3 - DNA, 219 bp, Artificial Sequence
  TPH2a riboprobe template
gccatgcagcccgcaatgatgatgttttccagtaaatactgggccaggagagggttgtccttggattctgctgtgccagaagatcatcagctacttggcagcttaacacaaaataaggctatcaaaagcgaggacaagaaaagcggcaaagagcccggcaaaggcgacaccacagagagcagcaagacagcagttgtgttctccttgaagaatgaagtt

SEQ ID NO: 4 - DNA, 219 bp, Artificial Sequence
  TPH2b riboprobe
gtgaaagcacttagactattccaggaaaaacatgtcaacatgcttcatatcgaatccaggcggtcccggcgaagaagttctgaagtcgaaatcttcgtggactgcgaatgtggcaaaacggaattcaatgagctcatccagttgctgaaatttcagaccaccattgtgaccctgaatccgcctgagagcatttggacggaggaagaagatctcgaggat

SEQ ID NO: 5 - DNA, 818 bp, Artificial Sequence
  TPH2c riboprobe
atgcagcccgcaatgatgatgttttccagtaaatactgggccaggagagggttgtccttggattctgctgtgccagaagatcatcagctacttggcagcttaacacaaaataaggctatcaaaagcgaggacaagaaaagcggcaaagagcccggcaaaggcgacaccacagagagcagcaagacagcagttgtgttctccttgaagaatgaagttggtgggctggtgaaagcacttagactattccaggaaaaacatgtcaacatgcttcatatcgaatccaggcggtcccggcgaagaagttctaagtcgaaatcttcgtggactgcgaatgtggcaaaacggaattcaatgagctcatccagttgctgaaatttcagaccaccattgtgaccctgaatccgcctgagagcatttggacggaggaagaagatctcgaggatgtgccgtggttccctcggaagatctctgagttagacagatgctctcaccgagtcctcatgtacggcaccgagcttgatgccgaccatccaggatttaaggacaatgtctatcgacagaggaggaagtattttgtggatgtggccatgggctataaatatggtcagcccattcccagggtcgagtacacagaagaagagactaaaacttggggtgttgtgttccgggagctctccaaactctacccgactcatgcttgccgggagtacctgaaaaacctccccctgctgaccaagtactgtggctacagggaagacaacgtgccgcaactggaagacgtctccatgtttctgaaagagcgatctggcttcacagtg

SEQ ID NO: 6 - DNA, 842 bp, Artificial Sequence
  TPH2-892 riboprobe
gaattcacggaagaagagattaagacctgggggaccatcttccgagagctaaacaaactctacccgacccacgcctgcagggagtacctcagaaacctccctttgctctcaaaatactgtggctatcgggaagacaacatcccgcaactggaggatgtctccaactttttaaaagaacgcactgggttttccatccgtcctgtggctggttacctctcaccgagagattttctgtcggggttagcctttcgagtctttcactgcactcagtatgtgagacacagttcagatcccctctacactccagagccagacacctgccatgaactcctaggccacgttcctctcttggctgaacccagttttgctcaattctcccaagaaattggcctggcttcccttggagcttcagaggagacagttcaaaaactggcaacgtgctactttttcactgtggagtttgggctgtgcaaacaagatggacagctgagagtctttggggccggcttgctttcttccatcagtgaactcaaacatgcactttctggacatgccaaagtcaagccctttgatcccaagattgcctgtaaacaggaatgtctcatcacgagcttccaggatgtctactttgtatctgagagctttgaagatgcaaaggagaagatgagagaatttgccaagaccgtgaagcgcccgtttggactgaagtacaacccgtacacacagagtgttcaggttctcagagacaccaagagcataactagtgccatgaatgagttgcggtagaccttgatgtcatcagtgatgccctcgctagggtcaccaggtggcccagtgtgtga

SEQ ID NO: 7 - DNA, 22 bp, Artificial Sequence
  Murine TPH2 forward primer mTPH2-514F
gaccaccattgtgaccctgaat

SEQ ID NO: 8 - DNA, 21 bp, Artificial Sequence
  Murine TPH2 forward primer mTPH2-1270F
ttcgtccatcggagaattgaa

SEQ ID NO: 9 - DNA, 22 bp, Artificial Sequence
  Murine TPH2 reverse primer mTPH2-585R
gaccaccattgtgaccctgaat

SEQ ID NO: 10 - DNA, 21 bp, Artificial Sequence
  Murine TPH2 reverse primer mTPH2-1344R
caggtcgtctttgggtcaaag

SEQ ID NO: 11 - DNA, 27 bp, Artificial Sequence
  Murine TPH2 probe mTPH2-565T
ttcttcctccgtccaaatgctctcagg

SEQ ID NO: 12 - DNA, 26 bp, Artificial Sequence
  Murine TPH2 probe mTPH2-1292T
catgctctttccgacaaggcgtgtgt

(19) United States
(12) Patent Application Publication
(10) Pub. No.: US 2007/0274916 A1
(43) Pub. Date: Nov. 29, 2007

(54) Method for Identifying Compounds that Affect Expression of Tryptophan Hydroxylase Isoform 2

(76) Inventor: Janet Clark, Matawan, NJ (US)

Correspondence Address: MERCK AND CO., INC, P O BOX 2000, RAHWAY, NJ, 07065-0907, US

(21) Appl. No.: 10/576,807

(22) PCT Filed: Oct. 20, 2004
(86) PCT No.: PCT/US04/34619

§ 371 Date: Apr. 21, 2006

Related U.S. Application Data
(60) Provisional application No. 60/514,268, filed on Oct. 24, 2003.

(51) Int. Cl. A61K 49/00; C12Q 1/68; A61K 31/573

(52) U.S. Cl. 424/9.2; 435/6; 514/171

(57) ABSTRACT

A method for identifying analytes which directly or indirectly affect tryptophan hydroxylase isoform 2 (TPH2) expression is described. The method enables glucocorticoid receptor modulators and &bgr;-hydroxysteroid dehydrogenase type 1 (&bgr,-HSD1) inhibitors to be screened for central nervous system penetrance and activity by determining their ability to regulate expression of TPH2. The method is particularly useful for identifying analytes which suppress glucocorticoid disruption of central serotonergic neurotransmission in the brain.

### BACKGROUND OF THE INVENTION

(1) Field of the Invention

The present invention relates to a method for identifying analytes which directly or indirectly affect tryptophan hydroxylase isoform 2 (TPH2) expression. The method enables glucocorticoid receptor modulators and 1 -hydroxysteroid dehydrogenase type 1 (β-HSD1) inhibitors to be screened for central nervous system penetrance and activity by determining their ability to modulate expression of TPH2. The method is particularly useful for identifying analytes which suppress glucocorticoid disruption of central serotonergic neurotransmission in the brain.

(2) Description of Related Art

The serotonin system mediates many central nervous facets of mood control and in regulating the sleep-wake cycle, feeding, thermoregulation, reproduction, arousal, awakefulness, anxiety, alcoholism, and drug abuse (Blundell, Neuropharmacol. 23: 1537-1551 (1984); Jacobs and Azmita, Physiol. Rev. 72: 165-229 (1992)). In the peripheral tissues, serotonin regulates vascular tone, gut motility, primary hemostasis, and cell-mediated immune responses (Veenstra-VanderWeele, Eur. J. Pharmacol. 410: 165- (2000)). Because serotonin is a key neurotransmitter in the central nervous system, dysregulation of serotonergic pathways has been implicated in the pathogenesis of many complex psychiatric diseases such as depression, schizophrenia, obsessive compulsive disorders, and suicide.

Tryptophan hydroxylase (TPH) is the rate limiting enzyme in the synthesis of the neurotransmitter serotonin (5-hydroxytryptamine (5-HT)). It also catalyzes the first, but not rate-limiting step, in the synthesis of the neurohormone melatonin. TPH is a member of the pterin-dependent aromatic amino acid monooxygenase family which includes phenylalanine hydroxylase and tyrosine hydroxylase. In the mammalian central nervous system, TPH mRNA expression is found in the cells of the dorsal raphe nucleus (DRN) and the median raphe nucleus (MRN). Both of these regions send ascending serotonin projections through the brain (Jacobs and Azmitia, J. Neurosci. 13: 5041-5055 (1992)). TPH mRNA expression is also found in the pineal gland where it is a non-rate limiting enzyme in the synthesis of the hormone melatonin, retina, the periphery in the enteric neurons of the gut, mast cells, platelets, and thyroid cells (Kuhn et al., J. Neurochem. 33: 15-21 (1979); Gershon, Ann. Rev. Neurosci. 4: 227-272 (1981); Champier et al., Life Sci. 60: 2191-2197 (1997); Kvetnansky and Sabban, Ann. NY Acad. Sci. 851: 342-356 (1998)).

Bethea et al. (Biol. Psychiat. 47: 562-576 (2000)) have reported modulation of TPH mRNA and protein levels by 17β-estradiol treatment in macaque monkey and guinea pig DRN (Bethea, ibid.; Lu et al., Endocrine 11: 257-267 (1999); Pecins-Thompson et al., J. Neurosci. 16: 7021-7029 (1996)). While changes in TPH mRNA or protein levels in response to 17β-estradiol treatment have not been reported in the rat, regulation of TPH protein, TPH enzyme activity, and TPH mRNA expression in the rat DRN have been shown to occur in response to glucocorticoids (Azmitia and. McEwen, Science 166: 1274-1276 (1969); Azmitia and McEwen, Brain Research 78: 291-302 (1974); Sze et al., J. Neurochem. 26: 169-173 (1976); Rastogi and Singhal, J. Neural Transm. 42: 63-71 (1978); Park et al., Neurosci. Res. 7, 76-80 (1989); Azmitia et al., J. Neurosci. 13: 5041-5055 (1993); Clark and Russo, Mol. Brain Res. 48: 346-354 (1997)).

Recently, Walther et al. (Science 299: 76 (2003)) had reported that there are two isoforms of TPH in the mouse, rat, and human genomes. The second isoform of TPH is TPH2 while the original TPH known in the art is now called TPH1. Based on RNA protection studies specific for each TPH isoform, TPH1 mRNA was found to be in the duodenum but not in the brain. In contrast, TPH2 mRNA was detected exclusively in the brain. Sugden in J. Neurochem. 86: 1308-1311 (2003) compared the relative expression of TPH1 and TPH2 mRNA in the rat pineal gland. Sugden found that TPH1 mRNA was about 100,000-fold more abundant than TPH2 mRNA and that TPH1 mRNA expression appeared to vary over the light:dark cycle whereas TPH2 mRNA expression showed no significant variation.

In light of the above, methods for specifically determining the level of TPH2 mRNA will be important in detecting and measuring abnormal serotonergic function in the brain, as well as in evaluating compounds which can directly or indirectly modulate TPH2 expression in the brain and thus, provide treatments for psychiatric diseases such as depression, schizophrenia, obsessive compulsive disorders, and prevention of suicide.

### BRIEF SUMMARY OF THE INVENTION

The present invention provides a method for identifying analytes which directly or indirectly affect tryptophan hydroxylase isoform 2 (TPH2) expression. The method enables glucocorticoid receptor modulators and 11β-hydroxysteroid dehydrogenase type 1 (β-HSD1) inhibitors to be screened for central nervous system penetrance and activity by determining their ability to modulate expression of TPH2, in particular, modulators and antagonists which interfere with glucocorticoid suppression of TPH2 expression and/or which stimulate TPH2 expression. Thus, in one aspect, the method is particularly useful for identifying analytes which suppress glucocorticoid disruption of central serotonergic neurotransmission in the brain.

Therefore, in one embodiment, the present invention provides a method for identifying an analyte which directly or indirectly modulates activity of a glucocorticoid receptor in the brain of an animal, which comprises (a) administering a glucocorticoid to a first animal and measuring an amount of tryptophan hydroxylase (TPH2) in the brain of the first animal; and (b) administering the glucocorticoid and the analyte to a second animal and measuring the amount of the TPH2 in the brain of the second animal wherein a change in the amount of the TPH2 in the brain of the second animal relative to the amount of the TPH2 in the first animal indicates that the analyte modulates the activity of the glucocorticoid receptor in the brain of the animal.

In particular aspects of the above embodiment, the glucocorticoid is selected from the group consisting of dexamethasone, prednisolone, and mifepristone.

In further aspects of the above, the analyte is selected from the group consisting of analytes having glucocorticoid agonist activity, analytes having glucocorticoid antagonist activity, and analytes having a mixture of agonist and antagonist activity.

In further still aspects of the above, the change in the TPH2 in the brain of the second animal is an increase in the amount of TPH2 mRNA.

In a further embodiment, the present invention provides a method for determining the effect of an analyte on an amount of tryptophan hydroxylase isoform 2 (TPH2) in the brain of an animal, which comprises (a) administering the analyte to the animal; and (b) measuring the amount of the TPH2 in the brain of the animal wherein a change in the amount of the TPH2 in the brain of the animal compared to the amount of the TPH2 in the brain of the animal without the analyte indicates that the analyte has an effect on the amount of the TPH2 in the brain of the animal.

In particular aspects of the above embodiment, the change in the amount of the TPH2 in the brain is either an increase in the amount of TPH2 mRNA or a decrease in the amount of TPH2 mRNA.

In a further still embodiment, the present invention provides a method for determining whether an analyte directly or indirectly affects the amount of tryptophan hydroxylase isoform 2 (TPH2) in the brain of an animal which has chronically elevated glucocorticoid levels, which comprises (a) providing an animal which has the chronically elevated glucocorticoid levels; (b) administering the analyte to the animal which has the chronically elevated glucocorticoid levels; and (c) measuring the amount of the TPH2 in the brain of the animal wherein an increase in the amount of the TPH2 in the brain of the animal compared to the amount of the TPH2 in the brain of the animal without the analyte indicates that the analyte has an effect on the amount of the TPH2 in the animal which has the chronically elevated glucocorticoid levels.

In particular aspects of the above embodiment, the chronically elevated glucocorticoid levels in the animal are the result of subjecting the animal to a stress inducing condition. In particular aspects, the stress inducing condition is chronic restraint stress or diet-induced obesity.

In further aspects of the above, the method screens for the analyte which is an inhibitor of 11β-hydroxysteroid dehydrogenase type 1 activity.

In further still aspects of the above, the affect of the analyte is an increase in the amount of the TPH2 in the brain of the animal.

In a further still embodiment, the present invention provides a method for identifying an analyte which directly or indirectly suppresses glucocorticoid levels in an animal which has chronically elevated glucocorticoid levels, which comprises (a) providing an animal having the chronically elevated glucocorticoid levels; (b) administering the analyte to the animal with the chronically elevated glucocorticoid levels; and (c) measuring an amount of tryptophan hydroxylase isoform 2 (TPH2) in the brain of the animal wherein an increase in the amount of the TPH2 in the brain of the animal compared to the amount of the TPH2 in the brain of the animal without the analyte indicates that the analyte suppresses the glucocorticoid levels in the animal which has the chronically elevated glucocorticoid levels.

In particular aspects of the above embodiment, the chronically elevated glucocorticoid levels in the animal are the result of subjecting the animal to a stress inducing condition. In particular aspects, the stress inducing condition is chronic restraint stress or diet-induced obesity.

In further aspects of the above, the method screens for the analyte which is an inhibitor of 11β-hydroxysteroid dehydrogenase type 1 activity.

In a further still embodiment, the present invention provides a method for determining whether an analyte is a full glucocorticoid agonist or antagonist or partial glucocorticoid agonist in the brain of an animal, which comprises (a) administering the analyte to a first animal and measuring an amount of tryptophan hydroxylase isoform 2 (TPH2) in the brain of the first animal; (b) administering a glucocorticoid to a second animal and measuring an amount of the TPH2 in the brain of the second animal; (c) administering the glucocorticoid and the analyte to a third animal and measuring an amount of the TPH2 in the brain of the third animal; and (d) comparing the amount of the TPH2 in the brains of the first, second, and third animals wherein (1) a decrease in the TPH2 in the brain of the first animal and a decrease in the TPH2 in the brain of the second animal which is not greater than the decrease in the TPH2 in the brain of the third animal indicates that the analyte is a full agonist, (2) an increase in TPH2 in the brain of the first animal and an increase in the TPH2 in the brain of the third animal compared to the TPH2 in the brain of the second animal indicates that the analyte is a full antagonist, and (3) a decrease in the TPH2 in the brain of the first animal and a decrease in the TPH2 in the brain of the second animal which is greater than the decrease in the TPH2 in the brain of the third animal indicates that the analyte is a partial agonist.

In particular aspects of the above embodiment, the glucocorticoid is selected from the group consisting of dexamethasone, prednisolone, and mifepristone.

In a further embodiment, the present invention provides a method for identifying an analyte which directly or indirectly suppresses glucocorticoid disruption of central serotonergic neurotransmission in the brain of an animal, which comprises: (a) administering a glucocorticoid to a first animal and measuring an amount of tryptophan hydroxylase (TPH2) in the brain of the first animal; and (b) administering the glucocorticoid and the analyte to a second animal and measuring an amount of the TPH2 in the brain of the second animal wherein a change in the amount of the TPH2 in the brain of the second animal relative to the amount of the TPH2 in the first animal indicates that the analyte suppresses the glucocorticoid disruption of the central serotonergic neurotransmission in the brain of the animal.

In particular aspects of the above embodiment, the glucocorticoid is selected from the group consisting of dexamethasone, prednisolone, and mifepristone.

In further aspects of the above, the change in the TPH2 in the brain of the second animal is an increase in the amount of the TPH2 mRNA.

In further still aspects of the above, the analyte is selected from the group consisting of analytes having glucocorticoid agonist activity, analytes having glucocorticoid antagonist activity, analytes having a mixture of agonist and antagonist activity, and analytes which inhibit 11β-hydroxysteroid dehydrogenase type-1 activity.

In any one of the above embodiments and aspects of the present invention, the animal is selected from the group consisting of mouse, guinea pig, rat, and primate and in further still aspects, the expression of the TPH2 is in a raphe slice preparation taken from the brain, particularly in the dorsal raphe nucleus of the brain.

In particular aspects of the above, the TPH2 is TPH2 mRNA.

In further aspects of any one of the above, the TPH2 is TPH2 mRNA and the TPH2 mRNA is measured by reverse transcription-polymerase chain reaction (RT-PCR). In a further still aspect, the RT-PCR is a real-time RT-PCR which uses an oligonucleotide probe comprising a nucleotide sequence complementary to a nucleotide sequence comprising the TPH2 mRNA. In further still aspects, the oligonucleotide probe is selected from the group consisting of SEQ ID NO:11 and SEQ ID NO:12 and the probe can be a riboprobe.

In further still aspects of any one of the above, the TPH2 is TPH2 RNA and the TPH2 mRNA is measured by in situ hybridization which uses an oligonucleotide probe comprising a nucleotide sequence complementary to a nucleotide sequence comprising the TPH2 mRNA. In further still aspects, the oligonucleotide probe comprises a nucleotide sequence selected from the group consisting of SEQ ID NO:3, SEQ ID NO:4, and SEQ ID NO:5.

In further still aspects of any one of the above, the TPH2 is measured using an antibody specific for the TPH2.

In a further embodiment, the present invention provides a kit for identifying an analyte which suppresses glucocorticoid disruption of central serotonergic neurotransmission in the brain of an animal, which comprises (a) at least one oligonucleotide probe which has a nucleotide sequence complementary to a nucleotide sequence comprising an mRNA encoding tryptophan hydroxyiase isoform 2 (TPH2); and (b) instructions for using the kit.

In particular aspects of the kit, the kit further includes a primer pair which flank the region of the nucleotide sequence comprising the mRNA which is complementary to the oligonucleotide probe.

In further aspects of the above, the kit further includes one or more components selected from the group consisting of a reverse transcriptase buffer, a reverse transcriptase enzyme, a polymerase chain reaction (PCR) buffer, dNTPs, and a thermostable DNA polymerase.

In further still aspects of the above, the kit further includes one or more regents for in situ hybridization and in further still aspects of the kit, the oligonucleotide probe is a riboprobe.

### BRIEF DESCRIPTION OF THE DRAWINGS

FIG. 1 shows an alignment of TPH1 and TPH2 amino acid sequences, SEQ ID NO:1 and SEQ ID NO:2, respectively. The Figure further shows the alignment of the real time RT-PCR and in situ hybridization biochemistry probes.

FIG. 2A shows the specificity of the TPH1 riboprobes for the in situ hybridization histochemistry. The Figure shows that TPH1 antisense but not sense riboprobes hybridized to murine DRN from estradiol-treated mice.

FIG. 2B shows the specificity of the TPH2 riboprobes for the in situ hybridization histochemistry. The Figure shows that non-overlapping TPH2a and TPH2b antisense riboprobes but not TPH2b sense riboprobe hybridized to murine DRN from estradiol-treated mice

FIG. 3A shows the distribution of TPH1 mRNA in coronal slice preparations of the murine DRN from untreated mice moving from the rostral to the caudal extent of the DRN. The hybridization probe was the TPH1-892 antisense riboprobe.

FIG. 3B shows the distribution of TPH2 mRNA in coronal slice preparations of the murine DRN from untreated mice moving from the rostral to the caudal extent of the DRN. The hybridization probe was the TPH2c antisense riboprobe.

FIG. 4A shows a coronal slice of murine brain at the level of the dorsal raphe showing the dissection used to make the raphe slice preparation for the real-time PCR analysis (panel from Paxinos and Watson, 1997). The brain tissue within the circle was used for the RT-PCR reactions (raphe slice preparation). PAG is periaqueductal grey, DR is dorsal raphe, scp is superior cerebellar peduncle, and MR is median raphe.

FIG. 4B shows that 17β-estradiol regulates expression of TPH1 mRNA in a murine raphe slice preparation as measured by real-time RT-PCR. TPH1 mRNA was induced 1.4 to 1.6 fold in the raphe slice from ovariectomized mice treated once daily for four days with 17β-estradiol (0.025 to 0.2 mg per kg body weight (mpk), subcutaneously (s.c.)).

FIG. 4C shows that 17β-estradiol at any dose had no apparent effect on expression of TPH2 mRNA in the murine dorsal raphe slice preparation from ovariectomized mice as measured by real-time RT-PCR. Mice were treated once daily for four days with 17β-estradiol (0.025 to 0.2 mpk, s.c.).

FIG. 5A shows that 17β-estradiol regulates expression of TPH1 mRNA in the murine DRN as measured by in situ hybridization histochemistry using the TPH1-892 antisense riboprobe. TPH1 mRNA was induced 3 fold in the DRN from ovariectomized mice treated once daily for four days with 17β-estradiol (0.2 mpk, s.c.).

FIG. 5B shows that 17β-estradiol (0.2 mpk, s.c.) had no apparent effect on expression of TPH2 mRNA in the murine DRN from ovariectomized mice as measured by in situ hybridization histochemistry using the TPH2c antisense riboprobe. Mice were treated once daily for four days with 17β-estradiol (0.2 mpk, s.c.).

FIG. 6 shows that glucocorticoids do not affect TPH1 mRNA levels in the murine raphe slice preparation as measured by real-time RT-PCR. TPH1 mRNA was induced 1.8 fold in the dorsal raphe of ovariectomized mice treated once daily for four days with 17β-estradiol (0.2 mpk, s.c.). Subcutaneous administration of dexamethasone or prednisolone had no effect on TPH1 message levels in the same experiment.

FIG. 7 shows a comparison of the effect of 17β-estradiol and the glucocorticoids dexamethasone, prednisolone, mifepristone (RU486) on TPH2 mRNA levels in the murine raphe slice preparation. Mice were treated once daily with either 0.2 mpk 17β-estradiol, 3 mpk dexamethasone, 10 mpk RU486, 10 mpk prednisolone, or vehicle control. The real-time PCR was performed using the mTPH2-514F and mTPH2-585R with the mTPH2-565T probe (TPH2-514 primer/probe set) or the mTPH2-1270F and mTPH2-1344R with the mTPH2-1292T probe (TPH2-1270 primer/probe set). The 17β-tradiol had no statistically significant effect on TPH2 mRNA expression compared to the vehicle whereas each of the glucocorticoids caused a significant reduction in TPH2 mRNA expression compared to the vehicle control.

FIG. 8 shows a comparison of the effect of 17β-estradiol and the glucocorticoids dexamethasone, prednisolone, mifepristone (RU486) on TPH2 m RNA levels in the murine raphe slice preparation as measured by real-time PCR using the TPH2-514 primer/probe set. Mice were treated as above except that the amount of RU486 was 20 mpk. The 17β-estradiol had no statistically significant effect on TPH2 mRNA expression compared to the vehicle whereas each of the glucocorticoids caused a significant reduction in TPH2 mRNA expression compared to the vehicle control.

FIG. 9 shows that mifepristone (RU486) antagonizes the effect of dexamethasone on murine TPH2 mRNA levels in the raphe slice preparation as measured by real-time RT-PCR. Administration of dexamethasone once daily for four days resulted in a 40% reduction in TPH2 mRNA levels (p<0.001) in the murine raphe slice. Co-administration of dexamethasone and mifepristone resulted in a 17% reduction in TPH2 mRNA levels which is statistically different from the dexamethasone effect (p<0.05) but not statistically different from the vehicle control value. Treatment with mifepristone alone showed a 28% reduction in TPH2 message levels (p<0.01).

FIG. 10 shows plasma corticosterone levels in male BKTO mice treated for 14 days with corticosterone.

FIG. 11 shows that in raphe slice preparation of the male BKTO rats treated with corticosterone for 14 days in FIG. 12, the level of TPH2 mRNA was significantly reduced.

FIG. 12 shows that the uterine weight measurements from ovariectomized female mice dosed once daily for four days with 17β-estradiol (0.2 mg/kg, s.c.) increased about 3.2 to 3.7 fold in response to the 17β-estradiol treatment (* p<0.001). This is the uterine weight data for the same experiment presented in 4b and 4c.

FIG. 13A shows representative autoradiographic images of TPH2 mRNA in the murine DRN of ovariectomized females dosed once daily for four days with 3 and 10 mg/kg dexamethasone as detected using in situ hybridization histochemistry.

FIG. 13B shows a densitometric analysis of autoradiographic images of TPH2 mRNA levels in the DRN of ovariectomized female mice subcutaneously administered 3 or 10 mg/kg dexamethasone. V is the vehicle for the dexamethasone. Subcutaneous administration of dexamethasone reduces TPH2 mRNA levels (28% at 3 mg/kg, 36% at 10 mg/kg) in the murine DRN (*p<0.001).

FIG. 14 shows that dexamethasone regulates TPH2 mRNA expression in murine raphe slice preparations from ovariectomized female mice as measured by real-time RT-PCR. Subcutaneous administration of dexamethasone at doses from 0.1 to 3 mg/kg once daily for four days resulted in a reduction in TPH2 mRNA levels from 23% at 0.1 mglkg to 44% at 3 mg/kg in the murine raphe slice preparation (&p<0.05; **p<0.005; *p≦0.001).

FIG. 15 shows that mifepristone antagonizes the effect of dexamethasone on murine TPH2 mRNA expression in raphe slice preparations from intact male mice as measured by real-time RT-PCR. Administration of dexamethasone to intact male mice once daily for four days resulted in a 26% reduction in TPH2 mRNA levels (*p<0.001) in the murine raphe slice. TPH2 mRNA levels were unchanged with coadministration of dexamethasone and mifepristone as compared with vehicle but were statistically different from the dexamethasone effect (&p<0.05).

### DETAILED DESCRIPTION OF THE INVENTION

The present invention provides a method and kit for identifying analytes which directly or indirectly affect tryptophan hydroxylase isoform 2 (TPH2) expression or levels in the brain of an animal. The method and kit enable glucocorticoid receptor modulators and 11β-hydroxysteroid dehydrogenase type 1 (β-HSD1) suppressors or inhibitors to be screened for central nervous system penetrance and activity by determining their ability to regulate expression of TPH2 in the brain of the animal. The method is particularly useful for identifying analytes which directly or indirectly suppress glucocorticoid disruption of central serotonergic neurotransmission in the brain and for designing protocols for treating neurological or psychiatric diseases or disorders such as depression (including psychotic depression), schizophrenia, obsessive compulsive disorders, other disorders such as sleep disorders, obesity, Type I diabetes mellitus, and appetite disorders; neurological or psychiatric disorders caused or induced by stress (including chronic stress), stress associated with a metabolic imbalance such as Type I diabetes mellitus, various kinds of cardiovascular diseases, or obesity, suicidal behavior; and, other diseases which have symptoms which correlate with depression involving glucocorticoid disruption of central serotonergic neurotransmission.

Recently, Walther et al. (Science 299: 76 (2003)) had reported that there are two isoforms of TPH in the mouse, rat, and human genomes. The first isoform (TPH1) is found in the duodenum but not in the brain and the second isoform (TPH2) is found exclusively in the brain. The different locations for the two isoforms suggests that there are two serotonin systems, the first uses TPH1 and is located in the peripheral tissues and the second uses TPH2 and is located in the brain. The dual serotonin system suggests that specific therapeutic treatments exclusively affecting either central or peripheral serotonin actions can be developed This is important for finding useful correlations between peripheral levels of serotonin and its metabolites and serotonin function in the central nervous system of patients suffering from psychiatric disorders such as depression, schizophrenia, obsessive compulsive disorders, and suicide. However, distinguishing TPH1 from TPH2 is problematic. Commercially available antibodies against TPH recognize both TPH isoforms making it impossible to distinguish the isoforms based on Western blot analyses or immunocytochemistry. In addition, it is difficult to determine which TPH isoform is regulated where changes in TPH protein or enzyme activity have been reported. Furthermore, it is not feasible to identify an isoform based on TPH activity levels because both activities would be present and not differentiated in any preparation of dorsal raphe tissue.

We have designed oligonucleotide probes which can be used to distinguish TPH2 mRNA from TPH1 mRNA and PCR primers which can be used in RT-PCR reactions to preferentially amplify TPH2 mRNA. As used herein, the term “mRNA” includes mRNA, hnRNA, or both.

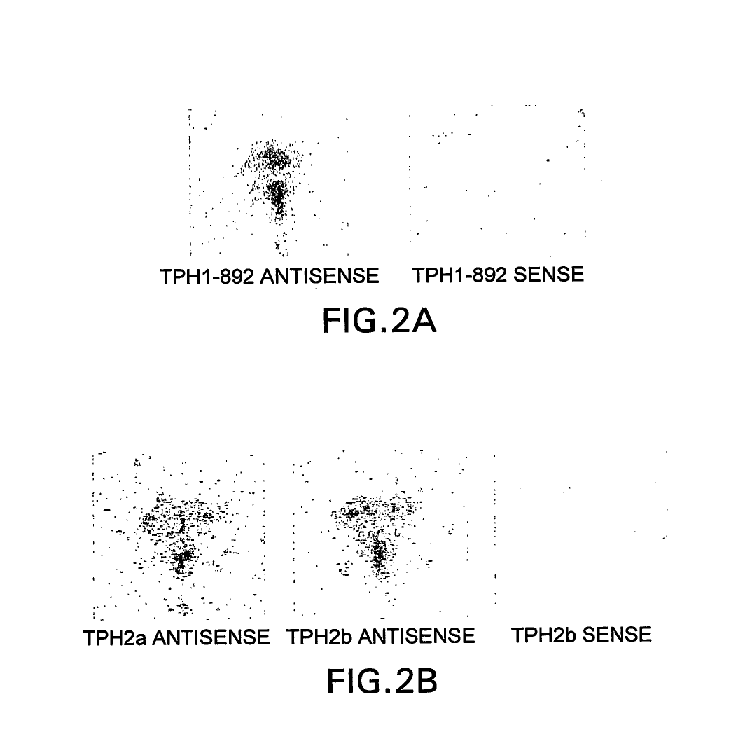

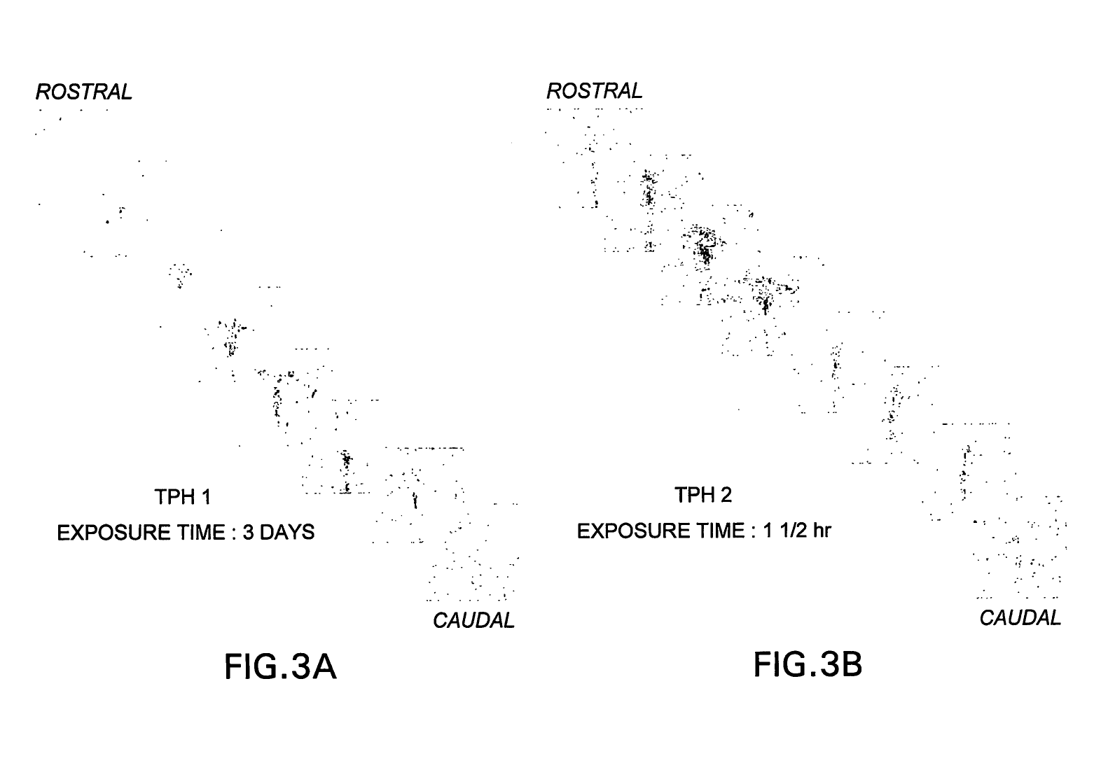

Using the antisense oligonucleotide probes, we discovered that TPH1 is expressed in the brain (See FIGS. 2A, 3A, and 6) and confirmed that TPH2 is expressed in the brain (See FIGS. 2B and 3B). This result was unexpected in light of Walther et al. (supra) which had shown by RNA protection studies that TPH1 was not expressed in the brain. Thus, we found that while TPH2 is robustly expressed in the DRN, there is detectable expression of TPH1 in the DRN by in situ hybridization and real-time RT-PCR.

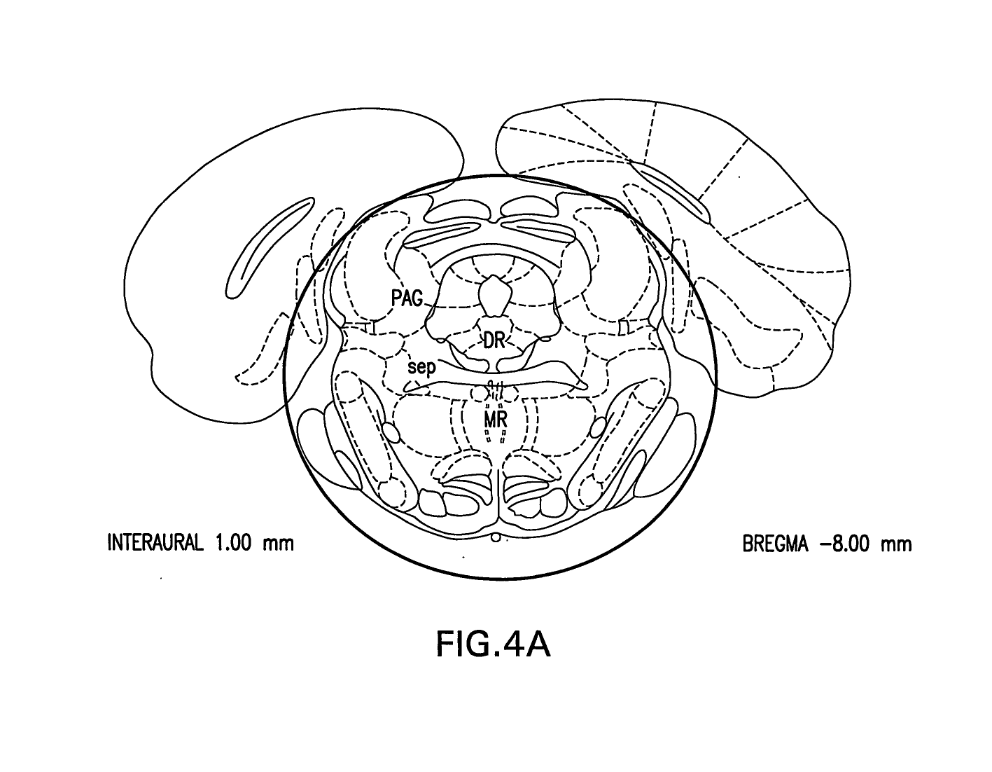

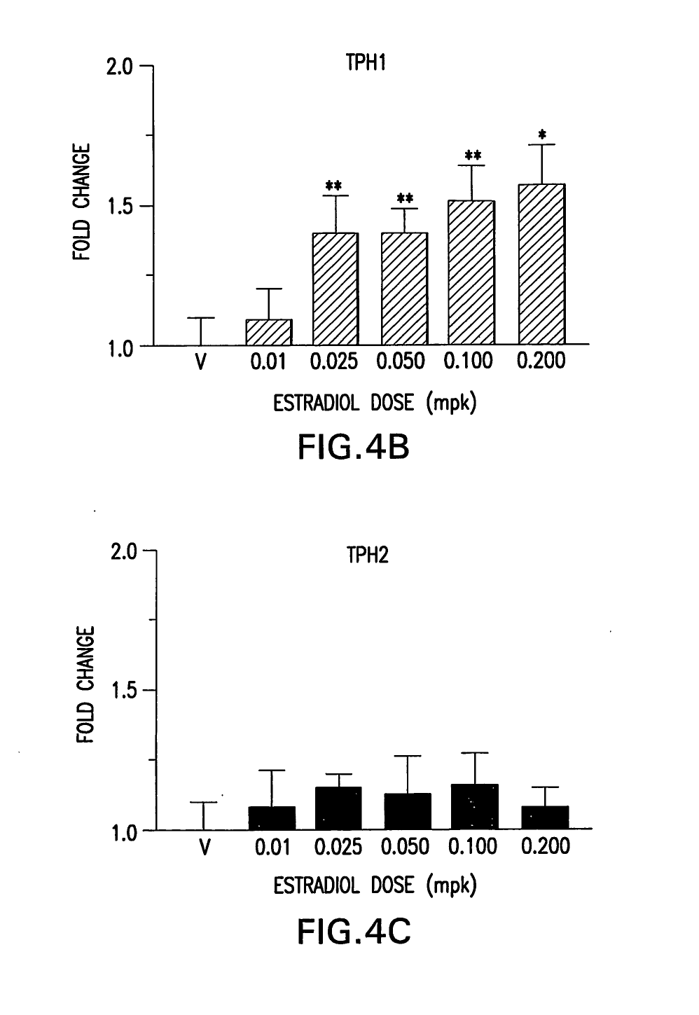

Using the antisense oligonucleotide probes for detecting TPH2 mRNA expression, we also discovered that TPH1 and TPH2 are differentially modulated in response to particular hormones. For example, we discovered that TPH1 and TPH2 mRNA expression in the murine dorsal raphe nucleus (DRN) is differentially regulated by 17β-estradiol. Using both real-time RT-PCR using TAQMAN technology (Applied Biosystems, Foster City, Calif.) and in situ hybridization histochemistry, 17β-estradiol increases TPH1 mRNA levels in the murine DRN through the estrogen receptor β while TPH2 mRNA levels remain unchanged (See FIGS. 5A and 5B (in situ hybridization); FIGS. 4B and 4C (RT-PCR)).

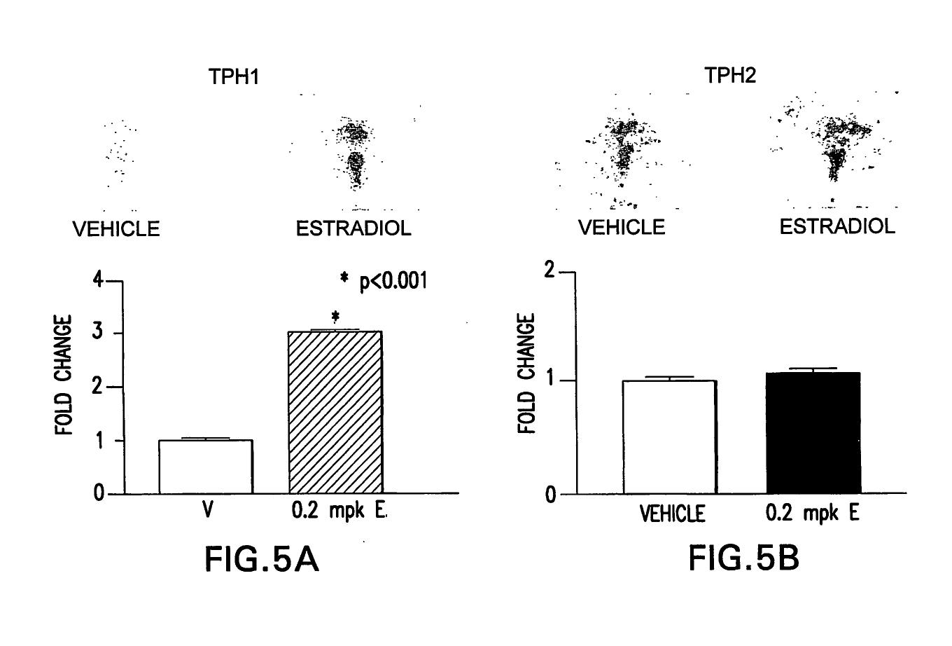

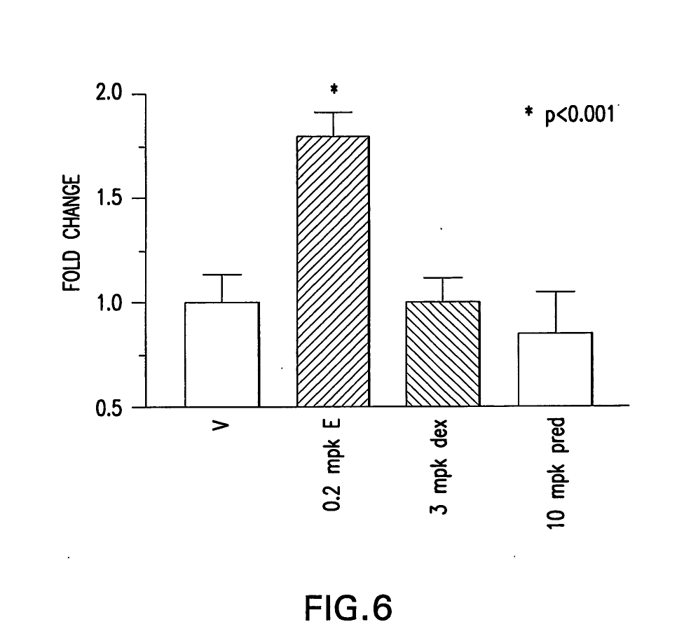

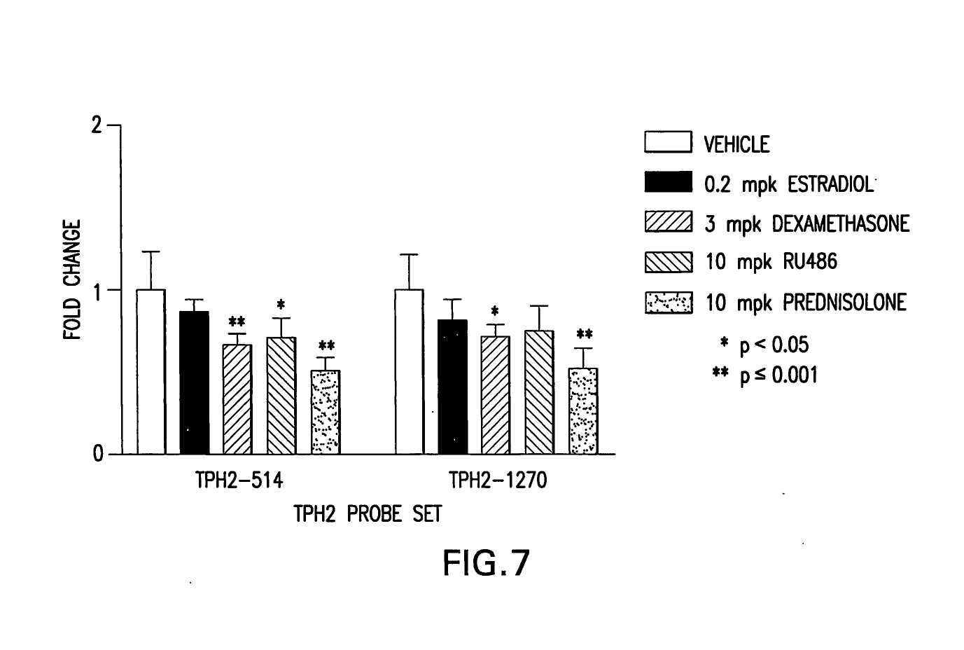

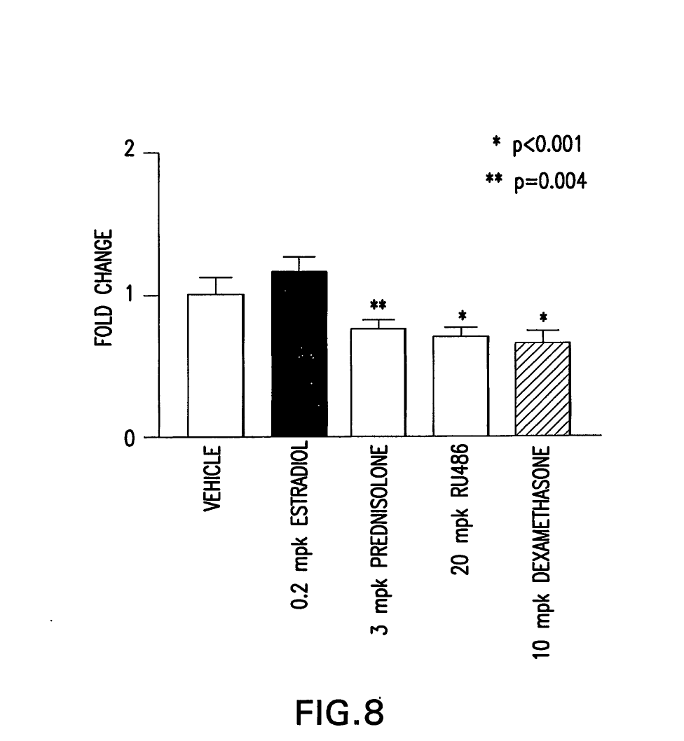

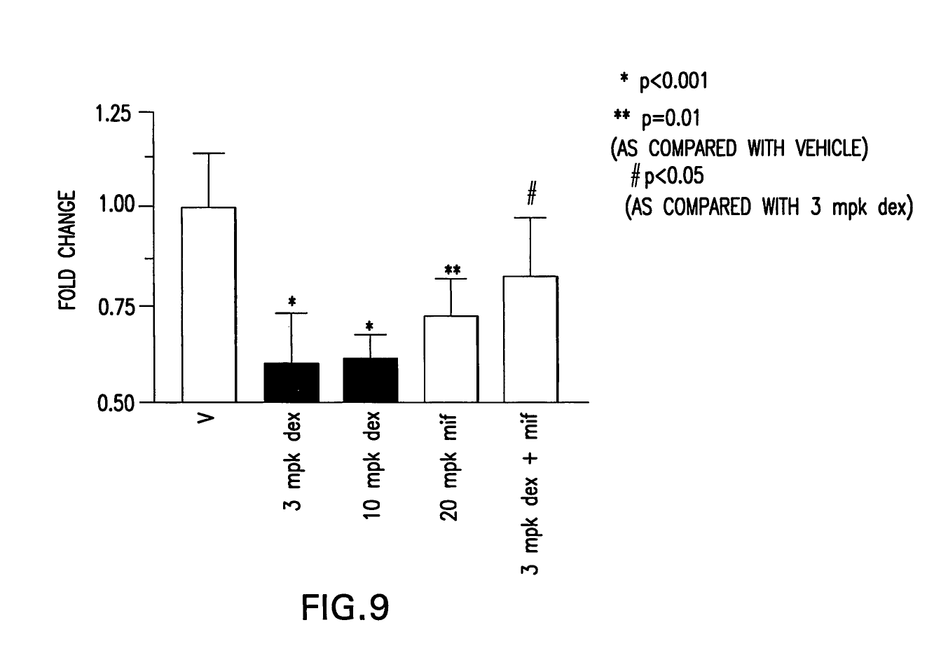

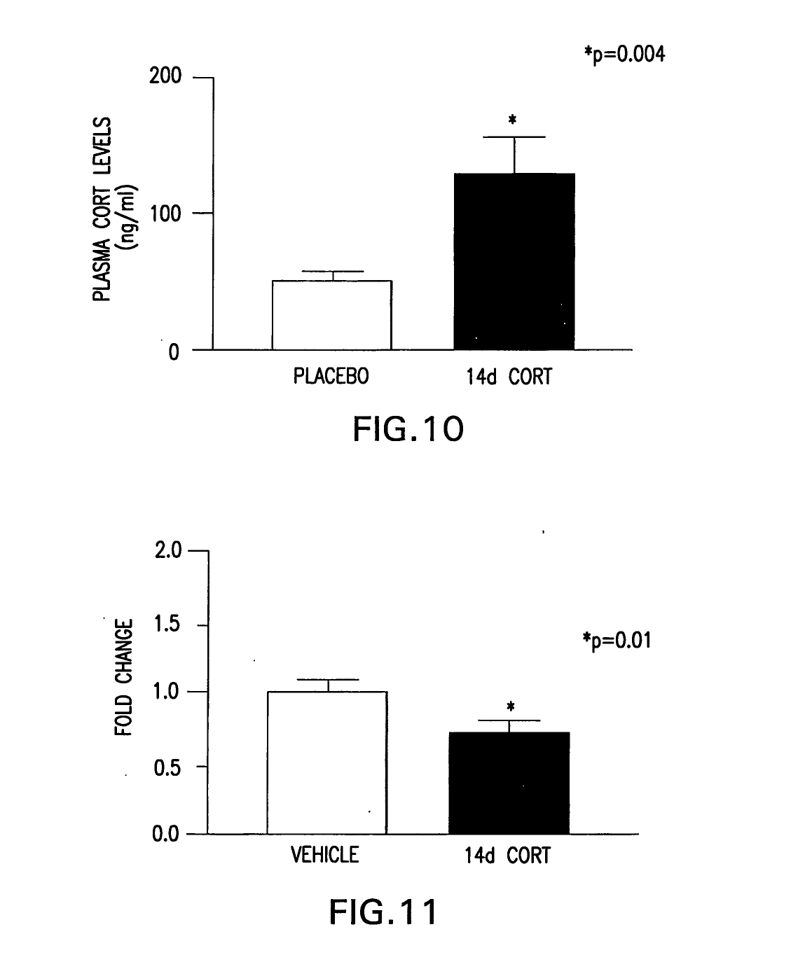

We further discovered that the mRNA expression of the TPH isoforms in the murine DRN are differentially regulated by glucocorticoids. Using real-time RT-PCR, TPH2 mRNA levels were reduced in murine raphe slice preparations in response to treatments with dexamethasone, prednisolone, or mifepristone (RU486) (See FIGS. 7 and 8) while TPH1 mRNA levels showed no detectable change in response to these treatments (See FIG. 6). These results indicate that TPH1 and TPH2 mRNA are differentially regulated by hormones in the murine DRN. Finally, as shown in FIG. 9, we found that while both dexamethasone and RU486 caused a decrease in TPH2 mRNA expression when administered separately, the RU486 antagonized the effect of dexamethasone when they were administered together. The glucocorticoid effect on TPH2 mRNA expression was observed in both ovariectomized female mice (See above) and in male mice (See FIGS. 10 and 11), thus the observed effect of glucocorticoids on TPH2 mRNA expression appears to be gender independent.

Therefore, the present invention provides oligonucleotide probes which preferentially hybridize to the nucleotide sequence encoding TPH2. The oligonucleotide probes can comprise natural or modified deoxyribonucleotides, ribonucleotides, or mixtures thereof. Preferably, the oligonucleotide probes are complementary or antisense to the nucleotide sequence of mRNA encoding TPH2 or cDNA encoding the TPH2. In particular embodiments, the oligonucleotide probes specific for the nucleotide sequence encoding TPH2.can be selected from the group consisting of SEQ ID NO:3, SEQ ID NO:4,SEQ ID NO:5, SEQ ID NO:11, and of SEQ ID NO:12. Other oligonucleotide probes specific for nucleic acids encoding TPH2 can be readily ascertained from the nucleic sequence encoding the human TPH2 as set forth in GenBank accession no. NM173353 or the mouse TPH2 as set forth in GenBank accession no. NM173391. It will be understood by one of skill in the art that probes will typically substantially bind target sequences lacking complete complementarity with the probe sequence depending upon the stringency of the hybridization conditions. The probes are preferably directly labeled with a label such as a radioactive isotope or indirectly labeled with a label such as biotin, to which a streptavidin complex can later bind. By assaying for the presence or absence of the probe, one can detect the presence or absence of the mRNA encoding TPH2. The probe can be labeled by any standard technique known in the art, such as radiolabeling, fluorescence labeling, and the like. Labels can include radioisotopes, FITC or other fluorochrome markers, enzymes, biotin, digoxigenin, fluorogenic quencher-donor dyes, or other molecules capable of detection. The probes are particularly useful for detecting mRNA encoding TPH2 in brain tissues, for example, the DRN, using in situ methods or in Northern hybridizations. Methods for making oligonucleotide probes, including riboprobes, and methods for performing in situ hybridizations and Northerns can be found in Sambrook et al., Molecular Cloning: A Laboratory Manual 2^{nd} Edition; Cold Spring Harbor Laboratory Press, Cold Spring Harbor, N.Y., (1989) or Sambrook and Russell, Molecular Cloning: A Laboratory Manual, 3rd Edition. Cold Spring Harbor Laboratory Press, Plainview, N.Y. (2001).

The present invention further provides oligonucleotide primers for PCR amplification of DNA encoding TPH2 or RT-PCR amplification of mRNA encoding TPH2. In particular embodiments, the forward oligonucleotide primers for PCR or RT-PCR can include the primers selected from the group consisting of SEQ ID NO:7 and of SEQ ID NO:8 and the reverse oligonucleotide primers for PCR or RT-PCR can include the primers selected from the group consisting of SEQ ID NO:9 and of SEQ ID NO:10. Other oligonucleotide PCR primers specific for nucleic acids encoding TPH2 can be readily ascertained from the nucleic sequence encoding the human TPH2 as set forth in GenBank accession no. NM173353 or the mouse TPH2 as set forth in GenBank accession no. NM173391. In a further embodiment, the PCR primers are used in an RT-PCR reaction to detect mRNA encoding TPH2. Methods for making cDNA and PCR and RT-PCR reactions are disclosed in Sambrook et al., Molecular Cloning: A Laboratory Manual 2nd Edition; Cold Spring Harbor Laboratory Press, Cold Spring Harbor, N.Y., (1989) or Sambrook and Russell, Molecular Cloning: A Laboratory Manual, 3rd Edition. Cold Spring Harbor Laboratory Press, Plainview, N.Y. (2001).

In a particularly preferred embodiment, the PCR primers are used in a real-time RT-PCR method such as the TAQMAN method. The real-time RT-PCR method enables mRNA encoding TPH2 to be detected and the amount or level of the TPH2 mRNA to be quantified. TAQMAN-based methods are well known in the art. In a real-time-based RT-PCR method, the PCR component of the reaction comprises a set of forward and reverse PCR primers and an oligonucleotide probe complementary to a sequence located between the sequences complementary to the primers. The probe is labeled at one end with a reporter dye and labeled at the other end with a quencher dye with the 3′ end block to prevent extension by the polymerase. Examples of reporter dyes include FAM, HEX, TET, JOE, or MAX and examples of quencher dyes include TAMRA or MGB. In the intact probe, the quencher dye suppresses fluorescence of the reporter dye, However, during PCR amplification, the 5′→3′ exonuclease activity of the polymerase digests the probe which releases the reporter dye from its close proximity to the quencher dye enabling the reporter dye to fluoresce. Because the fluorescence of the reporter dye is proportional to the amount template, the amount of TPH2 mRNA can be quantified. Therefore, the present invention provides methods for identifying analytes which directly or indirectly modulate expression of TPH2 mRNA in the brain of an animal which use the aforementioned oligonucleotide probes for detecting the TPH2 mRNA in the brain in situ and methods for detecting TPH2 mRNA expression in the brain by RT-PCR. The RT-PCR methods further include real-time RT-PCR methods for quantifying the expression of TPH2 mRNA in the brain, that is, determining the amount or level of TPH2 mRNA produced over time. The methods disclosed herein can be used in primary and secondary screening regimens to identify analytes which are glucocorticoid receptor modulators and 11β-hydroxysteroid dehydrogenase type 1 (β-HSD1) inhibitors and which are able to enter the brain and cause an increase in the expression or amount or levels of mRNA encoding TPH2. Such analytes are useful for treating psychiatric disorders which are a consequence of glucocorticoid disruption of the central serotonergic pathway. As used herein, the term “analyte” includes molecules, compounds, compositions, drugs, and formulations.

Therefore, in one embodiment, a method is provided for identifying an analyte which directly or indirectly modulates activity of a glucocorticoid receptor in the brain of an animal, which comprises administering a glucocorticoid to a first animal and measuring an amount of tryptophan hydroxylase (TPH2) in the brain of the first animal; and administering the glucocorticoid and the analyte to a second animal and measuring the amount of the TPH2 in the brain of the second animal. A change in the amount of the TPH2 in the brain of the second animal relative to the amount of the TPH2 in the first animal, particularly where the change is an increase in the amount of TPH2, indicates that the analyte modulates the activity of the glucocorticoid receptor in the brain of the animal.

In another embodiment, a method is provided for determining the effect of an analyte on an amount of tryptophan hydroxylase isoform 2 (TPH2) in the brain of an animal, which comprises administering the analyte to the animal; and measuring the amount of the TPH2 in the brain of the animal. A change in the amount of the TPH2 in the brain of the animal compared to the amount of the TPH2 in the brain of the animal without the analyte, particularly where the change is an increase in the amount of the TPH2, indicates that the analyte has an effect on the amount of the TPH2 in the brain of the animal.

The results shown in FIG. 9 indicated that the present invention further includes a method for determining whether an analyte is a full glucocorticoid agonist or antagonist or partial glucocorticoid agonist in the brain of an animal, which comprises (1) administering the analyte to a first animal and measuring an amount of tryptophan hydroxylase isoform 2 (TPH2) in the brain of the first animal; (2) administering a glucocorticoid to a second animal and measuring an amount of the TPH2 in the brain of the second animal; and (3) administering the glucocorticoid and the analyte to a third animal and measuring an amount of the TPH2 in the brain of the third animal. The amount of the TPH2 in the brains of the first, second, and third animals is compared. A decrease in the TPH2 in the brain of the first animal and a decrease in the TPH2 in the brain of the second animal which is not greater than the decrease in the TPH2 in the brain of the third animal indicates that the analyte is a full agonist. An increase in TPH2 in the brain of the first animal and an increase in the TPH2 in the brain of the third animal compared to the TPH2 in the brain of the second animal indicates that the analyte is a full antagonist. A decrease in the TPH2 in the brain of the first animal and a decrease in the TPH2 in the brain of the second animal which is greater than the decrease in the TPH2 in the brain of the third animal indicates that the analyte is a partial agonist.

In still further embodiment, a method is provided for identifying an analyte which directly or indirectly suppresses glucocorticoid disruption of central serotonergic neurotransmission in the brain of an animal, which comprises administering a glucocorticoid to a first animal and measuring an amount of tryptophan hydroxylase (TPH2) in the brain of the first animal; and administering the glucocorticoid and the analyte to a second animal and measuring an amount of the TPH2 in the brain of the second animal. A change in the amount of the TPH2 in the brain of the second animal relative to the amount of the TPH2 in the first animal, particularly where the change is an increase in the amount of TPH2 indicates that the analyte suppresses the glucocorticoid disruption of the central serotonergic neurotransmission in the brain of the animal.

In a typical protocol for the above method, female mice, which have been ovariectomized, or male mice are dosed subcutaneously at least once daily with vehicle control, a glucocorticoid such as dexamethasone, usually in an amount between about 3 and 20 mpk, a mixture of the dexamethasone and the analyte, or the analyte. In a typical assay, the mice are dosed over a time span of about four days. Approximately six hours following the fourth dose, mice are deeply anesthetized and brains removed from the skull and immediately frozen ventral side up on dry ice until analyzed. In some embodiments, the analyte is administered to the mice for one or more days before the glucocorticoid and analyte mixture is administered to the mice and in other embodiments, the analyte is administered to the mice in a mixture with the glucocorticoid one or more days after the mice have been administered the glucocorticoid. It is preferred to include vehicle controls when performing any one of the embodiments of the present invention disclosed herein.

The TPH2 which is measured can be either TPH2 protein, which is detected using antibodies specific for the TPH2, or TPH2 mRNA, which is detected using oligonucleotide probes specific for the TPH2 mRNA or RT-PCR primer/probe combinations which selectively amplify TPH2 mRNA.

TPH2 mRNA is preferably detected using in situ hybridization chemistry or RT-PCR, particularly real-time RT-PCR. Example 1 provides an example of using various probes and an in situ hybridization method to detect TPH2 mRNA in the dorsal raphe of mice. Examples 2 and 3 provide examples of primers and probe combinations and a real-time PCR method for detecting TPH2 mRNA in RNA extracted from the dorsal raphe.

In a variation of the above, a method is provided for determining whether an analyte directly or indirectly affects the amount of tryptophan hydroxylase isoform 2 (TPH2) in the brain of an animal which has chronically elevated glucocorticoid levels, which comprises providing an animal which has the chronically elevated glucocorticoid levels; and, administering the analyte to the animal which has the chronically elevated glucocorticoid levels. The amount of TPH2 in the brain of the animal is then measured. An increase in the amount of the TPH2 in the brain of the animal compared to the amount of the TPH2 in the brain of the animal without the analyte indicates that the analyte has an effect on the amount of the TPH2 in the animal which has the chronically elevated glucocorticoid levels.

In a further variation, a method is provided for identifying an analyte which directly or indirectly suppresses glucocorticoid levels in an animal which has chronically elevated glucocorticoid levels, which comprises providing an animal having the chronically elevated glucocorticoid levels; and, administering the analyte to the animal with the chronically elevated glucocorticoid levels. The amount of TPH2 in the brain of the animal is then measured. An increase in the amount of the TPH2 in the brain of the animal compared to the amount of the TPH2 in the brain of the animal without the analyte indicates that the analyte suppresses the glucocorticoid levels in the animal which has the chronically elevated glucocorticoid levels.

The above variations differ from the preceding embodiments in that the glucocorticoids are endogenous and the glucocorticoid levels are increased naturally by treating the animal with a stressor whereas in the preceding embodiments, the glucocorticoid levels are increased by administering the glucocorticoids exogenously. In a typical protocol for the above variations, stress is induced in female animals (rat, primate, mouse, guinea pig), which have been ovariectomized, or male animals (rat, primate, mouse, guinea pig) by applying stressor to the mice. For example, the stress can be chronic restraint stress, multiple stress, diet-induced obesity stress, stress in conjunction with diabetes, or the like. Magarinos and McEwen, Neurosci. 69: 83-88 (1995); Magarinos and McEwen, Proc. Natl. Acad. Sci. USA 97: 1156-11061 (2000) disclose methods for inducing stress in animals. Then, the mice are dosed subcutaneously at least once daily with vehicle control, or the analyte. In a typical assay, the mice are dosed over a time span of about four days. Approximately six hours following the fourth dose, mice are deeply anesthetized and brains removed from the skull and immediately frozen ventral side up on dry ice until analyzed as disclosed herein. The above variations are particularly suited for identifying analytes which are β-HSD1 inhibitors that modulate endogenous glucocorticoid levels such that the modulation induces an increase in the TPH2 levels in the brain of an animal. A review of β-HSD1 and its role as a tissue-specific amplifier of glucocorticoid action is provided by Seckl and Walker, Endocrinol. 142: 1371-1376 (2001).

In accordance with detecting TPH2 protein, there are provided antibodies having specific affinity for TPH2 or epitope thereof. The antibodies are useful for identifying TPH2 in situ. The term “antibodies” is intended to be a generic term which includes polyclonal antibodies, monoclonal antibodies, Fab fragments, single V_{H} chain antibodies such as those derived from a library of camel or llama antibodies or camelized antibodies (Nuttall et al., Curr. Pharm Biotechnol. 1: 253-263 (2000); Muyldermans, J. Biotechnol. 74: 277-302 (2001)), and recombinant antibodies. The term “recombinant antibodies” is intended to be a generic term which includes single polypeptide chains comprising the polypeptide sequence of a whole heavy chain antibody or only the amino terminal variable domain of the single heavy chain antibody (V_{H} chain polypeptides) and single polypeptide chains comprising the variable light chain domain (V_{L}) linked to the variable heavy chain domain (V_{H}) to provide a single recombinant polypeptide comprising the Fv region of the antibody molecule (scFv polypeptides)(See, Schmiedl et al., J. Immunol. Meth. 242: 101-114 (2000); Schultz et al., Cancer Res. 60: 6663-6669 (2000); Dubel et al., J. Immunol. Meth. 178: 201-209 (1995); and in U.S. Pat. No. 6,207,804 B1 to Huston et al.). Construction of recombinant single V_{H} chain or scFv polypeptides which are specific against an analyte can be obtained using currently available molecular techniques such as phage display (de Haard et al., J. Biol. Chem. 274: 18218-18230 (1999); Saviranta et al., Bioconjugate 9: 725-735 (1999); de Greeff et al., Infect. Immun. 68: 3949-3955 (2000)) or polypeptide synthesis. In further embodiments, the recombinant antibodies include modifications such as polypeptides having particular amino acid residues or ligands or labels such as horseradish peroxidase, alkaline phosphatase, fluors, and the like. Further still embodiments include fusion polypeptides which comprise the above polypeptides fused to a second polypeptide such as a polypeptide comprising protein A or G.

The antibodies specific for TPH2 can be produced by methods known in the art. For example, polyclonal and monoclonal antibodies can be produced by methods well known in the art, as described, for example, in Harlow and Lane, Antibodies: A Laboratory Manual. Cold Spring Harbor Laboratory Press: Cold Spring Harbor, N.Y. (1988). TPH2 or fragments thereof can be used as immunogens for generating such antibodies. Alternatively, synthetic peptides can be prepared (using commercially available synthesizers) and used as immunogens. Amino acid sequences can be analyzed by methods well known in the art to determine whether they encode hydrophobic or hydrophilic domains of the corresponding polypeptide. Altered antibodies such as chimeric, humanized, camelized, CDR-grafted, or bifunctional antibodies can also be produced by methods well known in the art. Such antibodies can also be produced by hybridoma, chemical synthesis or recombinant methods described, for example, in Sambrook et al., supra., and Harlow and Lane, supra. Both anti-peptide and anti-fusion protein antibodies can be used. (See, for example, Bahouth et al., Trends Pharmacol. Sci. 12: 338 (1991); Ausubel et al., Current Protocols in Molecular Biology (John Wiley and Sons, N.Y. (1989)).

The method of the present invention can be used for high throughput screening (ES) of analytes to identify analytes which are glucocorticoid receptor modulators (antagonists) or β-HSD1 inhibitors that cause an increase in the amount or levels of TPH2 mRNA in the brain. Alternatively, the method of the present invention can be used as an adjunct to high throughput screening method wherein analytes which have been identified by the high throughput screening method to be glucocorticoid receptor modulators or β-HSD1 inhibitors are then subjected to the method of the present invention to identify those analytes which cause an increase in the amount or levels of TPH2 mRNA in the brain. In either event, the method of the present invention identifies analytes which are glucocorticoid receptor modulators or β-HSD1 inhibitors that have central nervous system penetrance (that is, able to penetrate the blood-brain barrier). Often chemical entities with useful properties are generated by identifying a chemical compound (called a “lead compound”) with some desirable property or activity, creating variants of the lead compound, and evaluating the property and activity of those variant compounds. The current trend is to shorten the time scale for all aspects of drug discovery. Because of the ability to test large numbers quickly and efficiently, high throughput screening (HTS) methods are replacing conventional lead compound identification methods.

In one aspect, high throughput screening methods involve providing a library containing a large number of potential glucocorticoid modulators or β-HSD1 inhibitors which result in an increase in TPH2 mRNA expression in the brain (candidate compounds). Such “combinatorial chemical libraries” are then screened in one or more assays, to identify those library members particular chemical species or subclasses that display a desired characteristic activity. The compounds thus identified can serve as conventional “lead compounds” or can themselves be used as potential glucocorticoid modulators or HSD1 inhibitors.

Devices for the preparation of combinatorial libraries are commercially available (See, for example, 357 MPS, 390 MPS, Advanced Chem Tech, Louisville, Ky.; Symphony, Rainin, Woburn, Mass.; 433A Applied Biosystems, Foster City, Calif.; 9050 Plus, Millipore, Bedford, Mass.). A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (See, for example, ComGenex, Princeton, N.J.; Asinex, Moscow, Russia; Tripos, Inc., St. Louis, Mo.; ChemStar, Ltd, Moscow, Russia; 3D Pharmaceuticals, Exton, Pa.; Martek Biosciences, Columbia, Md.).

Any of the assays described herein are amenable to high throughput screening, including TPH2-riboprobe based hybridization and TPH2-primer based RT-PCR methods. As described above, the glucocorticoid modulators or β-HSD1 inhibitors are preferably screened by the methods disclosed herein. High throughput systems for such screening are well known to those of skill in the art. Thus, for example, U.S. Pat. No. 5,559,410 discloses high throughput screening methods for protein binding, while U.S. Pat. Nos. 5,576,220 and 5,541,061 disclose high throughput methods of screening for ligand/antibody binding.

In addition, high throughput screening systems are commercially available (See, for example, Zymark Corp., Hopkinton, Mass.; Air Technical Industries, Mentor, Ohio; Beckman Instruments, Inc. Fullerton, Calif.; Precision Systems, Inc., Natick, Mass.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols. Thus, for example, Zymark Corp. provides technical bulletins describing screening systems for detecting the modulation of gene transcription, ligand binding, and the like.

In light of the above, the present invention further provides a kit for identifying an analyte which suppresses glucocorticoid disruption of central serotonergic neurotransmission in the brain of an animal, which comprises at least one oligonucleotide probe which has a nucleotide sequence complementary to a nucleotide sequence comprising an mRNA encoding tryptophan hydroxylase isoform 2 (TPH2); and instructions for using the kit. In particular aspects of the kit, the kit further includes a primer pair which flank the region of the nucleotide sequence comprising the mRNA which is complementary to the oligonucleotide probe. In further aspects of the above, the kit further includes one or more components selected from the group consisting of a reverse transcriptase buffer, a reverse transcriptase enzyme, a polymerase chain reaction (PCR) buffer, dNTPs, and a thermostable DNA polymerase. In further still aspects of the above, the kit further includes one or more regents for in situ hybridization and in further still aspects of the kit, the oligonucleotide probe is a riboprobe. Examples or probes and primers which can be included in the kit are provided in Examples 1-3.

The following examples are intended to promote a further understanding of the present invention.

### EXAMPLE 1

In this example, TPH1 and TPH2 mRNA expression in the DRN was measured using in situ hybridization histochemistry methods. This example also shows that 17β-estradiol had an inhibitory effect on TPH1 mRNA expression in the DRN and no effect on TPH2 mRNA expression.

The animal and treatment groups consisted of the following. Female mice (13 to 16 wks of age) were ovariectomized (C57BL/6s from Charles River). Animals were fed a soy-free rodent chow and allowed a minimum of one week to recuperate from surgery and shipping. Mice were dosed subcutaneously (s.c.) in the morning (once daily for 4 days) with 0.1 cc of vehicle (sesame oil) or compound (3, 10, or 20 mg per kg body weight (mpk) of dexamethasone, RU486, or prednisolone as indicated or 0.2 mpk 17β-estradiol as indicated). Approximately six hours following the fourth dose, mice were deeply anesthetized with ketamine/xylazine and brains were removed from the skull and immediately frozen ventral side up on dry ice.

Molecular biology methods include the methods in Sambrook et al., Molecular Cloning: A Laboratory Manual 2^{nd} Edition; Cold Spring Harbor Laboratory Press, Cold Spring Harbor, N.Y., (1989) or Sambrook and Russell, Molecular Cloning: A Laboratory Manual, 3rd Edition. Cold Spring Harbor Laboratory Press, Plainview, N.Y. (2001).

Three TPH2-riboprobes were used to perform in situ hybridization on cryostat-cut 16 μm thick coronal murine brain sections. Two of the TPH2 riboprobe templates were non-overlapping (TPH2a riboprobe and TPH2b riboprobe) and were produced from a cDNA template comprising nucleotides 134 to 349 and 359 to 577, respectively, of the nucleotide sequence encoding TPH2 (See GenBank accession no. AY090565). A third TPH2 riboprobe (TPH2c riboprobe) template was produced from a cDNA template comprising nucleotides 134 to 952 of the nucleotide sequence encoding TPH2. A TPH1 riboprobe (TPH1-892) produced from a cDNA template comprising nucleotides 595-1437 of the TPH1 cDNA (GenBank accession no. J04758) was also used. A sense and an antisense version of each probe was synthesized using ³³P-labeled UTP (NEN-Dupont, Boston, Mass.) incorporated into cRNA. The probes were transcribed using cDNA templates containing RNA polymerase sequence extensions for T7 (antisense) and T3 (sense). A NUCTRAP push column (Stratagene, La Jolla, Calif.) was used for removal of unincorporated nucleotides. The riboprobe templates were amplified from mouse raphe slice cDNA using primers against the mouse sequence.

The TPH2a riboprobe template sequence was 5′-GCC ATG CAG CCC GCA ATG ATG ATG TTT TCC AGT AAA TAC TGG GCC AGG AGA GGG TTG TCC TTG GAT TCT GCT GTG CCA GAA GAT CAT CAG CTA CTT GGC AGC TTA ACA CAA AAT AAG GCT ATC AAA AGC GAG GAC AAG AAA AGC GGC AAA GAG CCC GGC AAA GGC GAC ACC ACA GAG AGC AGC AAG ACA GCA GTT GTG TTC TCC TTG AAG AAT GAA GTT-3′ (SEQ ID NO:3).

The TPH2b riboprobe template sequence was 5′-GTG AAA GCA CTT AGA CTA TTC CAG GAA AAA CAT GTC AAC ATG CTT CAT ATC GAA TCC AGG CGG TCC CGG CGA AGA AGT TCT GAA GTC GAA ATC TTC GTG GAC TGC GAA TGT GGC AAA ACG GAA TTC AAT GAG CTC ATC CAG TTG CTG AAA TTT CAG ACC ACC ATT GTG ACC CTG AAT CCG CCT GAG AGC ATT TGG ACG GAG GAA GAA GAT CTC GAG GAT-3′ (SEQ ID NO:4)

The TPH2c riboprobe template sequence was 5′-ATG CAG CCC GCA ATG ATG ATG TTT TCC AGT AAA TAC TGG GCC AGG AGA GGG TTG TCC TTG GAT TCT GCT GTG CCA GAA GAT CAT CAG CTA CTT GGC AGC TTA ACA CAA AAT AAG GCT ATC AAA AGC GAG GAC AAG AAA AGC GGC AAA GAG CCC GGC AAA GGC GAC ACC ACA GAG AGC AGC AAG ACA GCA GTT GTG TTC TCC TTG AAG AAT GAA GTT GGT GGG CTG GTG AAA GCA CTT AGA CTA TTC CAG GAA AAA CAT GTC AAC ATG CTT CAT ATC GAA TCC AGG CGG TCC CGG CGA AGA AGT TCT AAG TCG AAA TCT TCG TGG ACT GCG AAT GTG GCA AAA CGG AAT TCA ATG AGC TCA TCC AGT TGC TGA AAT TTC AGA CCA CCA TTG TGA CCC TGA ATC CGC CTG AGA GCA TTT GGA CGG AGG AAG AAG ATC TCG AGG ATG TGC CGT GGT TCC CTC GGA AGA TCT CTG AGT TAG ACA GAT GCT CTC ACC GAG TCC TCA TGT ACG GCA CCG AGC TTG ATG CCG ACC ATC CAG GAT TTA AGG ACA ATG TCT ATC GAC AGA GGA GGA AGT ATT TTG TGG ATG TGG CCA TGG GCT ATA AAT ATG GTC AGC CCA TTC CCA GGG TCG AGT ACA CAG AAG AAG AGA CTA AAA CTT GGG GTG TTG TGT TCC GGG AGC TCT CCA AAC TCT ACC CGA CTC ATG CTT GCC GGG AGT ACC TGA AAA ACC TCC CCC TGC TGA CCA AGT ACT GTG GCT ACA GGG AAG ACA ACG TGC CGC AAC TGG AAG ACG TCT CCA TGT TTC TGA AAG AGC GAT CTG GCT TCA CAG TG-3′ (SEQ ID NO:5).

The TPH2-892 riboprobe template sequence was 5′-GAA TTC ACG GAA GAA GAG ATT AAG ACC TGG GGG ACC ATC TTC CGA GAG CTA AAC AAA CTC TAC CCG ACC CAC GCC TGC AGG GAG TAC CTC AGA AAC CTC CCT TTG CTC TCA AAA TAC TGT GGC TAT CGG GAA GAC AAC ATC CCG CAA CTG GAG GAT GTC TCC AAC TTT TTA AAA GAA CGC ACT GGG TTT TCC ATC CGT CCT GTG GCT GGT TAC CTC TCA CCG AGA GAT TTT CTG TCG GGG TTA GCC TTT CGA GTC TTT CAC TGC ACT CAG TAT GTG AGA CAC AGT TCA GAT CCC CTC TAC ACT CCA GAG CCA GAC ACC TGC CAT GAA CTC CTA GGC CAC GTT CCT CTC TTG GCT GAA CCC AGT TTT GCT CAA TTC TCC CAA GAA ATT GGC CTG GCT TCC CTT GGA GCT TCA GAG GAG ACA GTT CAA AAA CTG GCA ACG TGC TAC TTT TTC ACT GTG GAG TTT GGG CTG TGC AAA CAA GAT GGA CAG CTG AGA GTC TTT GGG GCC GGC TTG CTT TCT TCC ATC AGT GAA CTC AAA CAT GCA CTT TCT GGA CAT GCC AAA GTC AAG CCC TTT GAT CCC AAG ATT GCC TGT AAA CAG GAA TGT CTC ATC ACG AGC TTC CAG GAT GTC TAC TTT GTA TCT GAG AGC TTT GAA GAT GCA AAG GAG AAG ATG AGA GAA TTT GCC AAG ACC GTG AAG CGC CCG TTT GGA CTG AAG TAC AAC CCG TAC ACA CAG AGT GTT CAG GTT CTC AGA GAC ACC AAG AGC ATA ACT AGT GCC ATG AAT GAG TTG CGG TAG ACC TTGA TGT CAT CAG TGA TGC CCT CGC TAG GGT CAC CAG GTG GCC CAG TGT GTG A-3′ (SEQ ID NO:6).

The In situ hybridization was performed as follows. Slides containing 16 μm thick coronal dorsal raphe sections of the mouse brain (See FIG. 4A) were briefly post-fixed in 4% formaldehyde in 1×PBS buffer (Ambion, Austin, Tex.), rinsed in 1×PBS, acetylated with 0.25% acetic anhydride in 0.1M triethanolamine, and rinsed in 2× saline sodium citrate (SSC) (2×SSC: 0.3M NaCl, 0.03M sodium citrate). After rinses and ethanol dehydration, sections were hybridized overnight at 55° C. with 5×10⁴ cpm probe/μL. The following morning, sections were washed twice in 2×SSC at room temperature, treated with ribonuclease A (RNase A) at 37° C. for 30 minutes, washed once in 2×SSC, once in 1×SSC at room temperature, and twice at 0.2×SSC at 45° C. or 65° C., as noted. Sections were dehydrated using ethanol, and apposed to I-sensitive film (BIOMAX MR, NEN-Dupont) for 1 to 3 hours at room temperature. Autoradiograph images of midbrain sections were captured using a CCD video camera (Dage-MTI Inc., Michigan City, Ind.) fitted with NIKON lenses (Nikon Canada, Inc.), and the Scion Image Program.

FIGS. 2A and 2B show that the TPH1 and TPH2 riboprobes which comprised nucleotide sequences antisense to the mRNA were specific for detecting mRNA encoding TPH1 and TPH2, respectively, by in situ hybridization in DRN slices from ovariectomized mice treated with 17β-estradiol. To test cross reactivity of the TPH2b riboprobe with TPH1 message, COS-7 cells were transfected with the coding sequence for either TPH1 or TPH2 subcloned into pcDNA-3.1. The TPH2b riboprobe hybridized to COS-7 cells overexpressing TPH2 mRNA but no hybridization was detected in COS-7 cells overexpressing TPH1 mRNA. To test cross reactivity of the TPH1-892 riboprobe with TPH2 message, COS-7 cells were transfected with the coding sequence for either TPH1 or TPH2 subcloned into pcDNA3.1. The TPH1-892 riboprobe hybridized to COS-7 cells overexpressing TPH1 mRNA but no hybridization was detected in COS-7 cells overexpressing TPH2 mRNA.

FIGS. 3A and 3B show that the amount and distribution of TPH1 and TPH2 mRNA in the DRN of ovariectomized mice are distinguishable. FIGS. 3A and 3B are autoradiographs of coronal midbrain slice preparations moving from the rostral to the caudal extent of the DRN of the ovariectomized mouse hybridized to either the TPH1-892 (FIG. 3A) or the TPH2c antisense riboprobe (FIG. 3B). FIGS. 3A and 3B show that expression of TPH2 mRNA was significantly greater in the DRN than expression of TPH1 in mRNA in the DRN (compare the 3 day exposure of the DRN slices hybridized with the TPH1-892 antisense riboprobe compared to the 1.5 hour exposure of the DRN slices hybridized with the TPH2c antisense riboprobe).

FIGS. 5A and 5B show that while 17β-estradiol increases TPH1 mRNA levels in the DRN of ovariectomized mice, it has no apparent effect on TPH2 mRNA levels. This can be seen by comparing the autoradiograph of a DRN slice from an ovariectomized mouse treated with 17β-estradiol and hybridized to the TPH1-892 antisense riboprobe to a DRN slice from an ovariectomized mouse treated with 17β-estradiol and hybridized to the TPH2c antisense riboprobe.

### EXAMPLE 2

In this example, the effect of dexamethasone, RU486, estradiol, prednisolone, or vehicle on murine TPH2 mRNA expression was measured using real-time quantitative PCR methods. In general, real-time PCR is more sensitive at detecting mRNA expression than the in situ hybridization method described in Example 1. This example shows that dexamethasone, RU486, 17β-estradiol, and prednisolone each had an inhibitory effect on TPH2 mRNA expression in the raphe slice preparation and no effect on TPH1 mRNA expression. This example further shows that 17β-estradiol had no detectable effect on TPH2 mRNA expression. In addition, this example shows that RU486 is a mixed or partial agonist in that alone, it inhibited TPH2 mRNA expression, but when mixed with dexamethasone, it antagonized dexamethasone inhibition of TPH2 mRNA expression.

Female mice (13 to 16 wks of age) were ovariectomized (C57BL/6s from Charles River; ER Knockout animals from Taconic). Animals were fed a soy-free rodent chow and allowed a minimum of one week to recuperate from surgery and shipping. Mice were dosed subcutaneously in the morning (once daily for 4 days) with 0.1 cc of vehicle (sesame oil) or compound (3, 10, or 20 mpk dexamethasone, RU486, estradiol, or prednisolone as indicated). Approximately six hours following the fourth dose, mice were deeply anesthetized with ketamine/xylazine and brains were removed from the skull, placed ventral side up in a mouse brain block on ice, and ice-cold razor blades were inserted into the block at 1 mm intervals. The caudal extent of the hypothalamus was used as an anatomical marker for the placement of the first razor blade, and 4 blades were placed in sequential slots, caudally. The four sections were examined and the two that encompassed the greatest extent of the dorsal raphe were placed in a tube containing RNALATER (Ambion, Austin, Tex.) and placed at 4° C. overnight. Brain slices were removed from RNALATER after 24 hours and stored at −80° C. in fresh tubes.

Molecular biology methods include the methods in Sambrook et al., Molecular Cloning: A Laboratory Manual 2^{nd} Edition; Cold Spring Harbor Laboratory Press, Cold Spring Harbor, N.Y., (1989) or Sambrook and Russell, Molecular Cloning: A Laboratory Manual, 3rd Edition. Cold Spring Harbor Laboratory Press, Plainview, N.Y. (2001).

Two TPH2 primer and probe sets were generated for use in detecting TPH2 by real-time RT-PCR. Murine TPH2 forward primers were mTPH2-514F and mTPH2-1270F, their corresponding sequences are 5′-GACCACCATTGTGACCCTGAAT-3′ (SEQ ID NO:7; corresponding to nucleotides 514 to 535 of the nucleotide sequence encoding TPH2) and 5′-TTCGTCCATCGGAGAATTGAA-3′ (SEQ ID NO:8; corresponding to nucleotides 1270 to 1290 of the nucleotide sequence encoding TPH2), respectively. The murine TPH2 reverse primers were mTPH2-585R and mTPH2-1344R, their corresponding sequences are 5′-GACCACCATTGTGACCCTGAAT-3′ (SEQ ID NO:9; corresponding to nucleotides 567 to 585 of the nucleotide sequence encoding TPH2) and 5′-CAGGTCGTCTTTGGGTCAAAG-3′ (SEQ ID NO:10; corresponding to nucleotides 1324 to 1344 of the nucleotide sequence encoding TPH2), respectively. The murine TPH2 probes were mTPH2-565T and mTPH2-1292T, their corresponding sequences are: 5′-TTCTTCCTCCGTCCAAATGCTCTCAGG-3′ (SEQ ID NO:11; corresponding to nucleotides 539 to 565 of the nucleotide sequence encoding TPH2) and 5′-CATGCTCTTTCCGACAAGGCGTGTGT-3′ (SEQ ID NO:12; corresponding to nucleotides 1292 to 1317 of the nucleotide sequence encoding TPH2), respectively. The probes were labeled at the 5′ end with FAM and the 3′ end with TAMRA.

Isolation of total RNA from mouse raphe slices for real-time PCR analysis using the TAQMAN ABI PRISMS 7700 Detection System, Applied Biosystems, Foster City, Calif. was as follows. Samples were stored in RNALATER at 4° C. overnight followed by removal of RNALATER and storage at −80° C. until isolation of the total RNA (2 slices weigh 25 to 50 mg). Slices were removed from −80° C. and placed in 1.0 mL TRIZOL Reagent in FASTPREP processing tubes. Slices were homogenized with one pass at setting 6 for 30 seconds in FASTPREP 120 homogenizer using LYSING MATRIX D tubes with bead matrix followed by 20 seconds at setting 6 after all samples had been processed. Samples were set at room temperature for 5 minutes to allow for complete dissociation of nucleoprotein complexes followed by centrifugation of the samples at 12,000×g for 5 minutes at 4° C. Homogenates were transferred to 1.5 mL; microcentrifuge tubes and 100 μL BCP (Bromo-3-chloropropane) was added. Samples were vortexed for 15 seconds and set at room temperature for 2 to 3 minutes. Samples were centrifuged at 12,000×g for 15 minutes at 4° C. The aqueous layer was removed and placed in a new RNAse-free sterile 1.5 mL microcentrifuge tube. Five μL of 5 mg/μL glycogen was added to each sample and the samples were vortexed. Five hundred μL of isopropanol was added to each sample. The samples were vortexed for 15 seconds, set at room temperature 10 minutes, followed by centrifugation at 12,000×g for 15 minutes at 4° C. Supernatants were decanted and pellets washed with 500 μL ice cold 75% ethanol. Samples were centrifuged at 12,000×g for 15 minutes at 4° C., ethanol decanted, and pellets air dried for 10 minutes. Pellets were resuspended in 30 μL prewarmed RNASECURE (60° C.), and heated at 60° C. for 10 min. Samples were stored at −80° C. until DNAse treatment and cDNA synthesis.

DNase treatment and cDNA preparation with wt mouse raphe slice total RNA for TAQMAN analysis. DNase treatment using DNA-free kit (Ambion). Five μg of the total RNA sample was aliquotted to each well of a 96 well plate. An 1× DNase I solution was added to each sample (DNase I buffer, DNase I, H₂O). Reactions were mixed and incubated at 37° C. for 30 minutes. Reactions were inactivated by addition of DNase inactivation reagent beads, mixed well at room temperature for 3 minutes and centrifuged at 2,500 RPM for 1 minutes at 4° C. (25 μL reactions were run and inactivated with 1/10 volume of inactivation reagent).

Reverse Transcription. A 10 μL of the DNase I-treated total RNA was added to 40 μL of 1× reverse transcription reaction mix (DEPC treated H₂O, RT buffer, MgCl₂, dNTP mix, random hexamers, RNase inhibitor, and MULTISCRIBE RT) and incubated at 25° C. for 10 minutes, 48° C. for 30 minutes, and 95° C. for 5 minutes. Reverse transcription was halted by the addition of EDTA. Samples were transferred to a storage plate and stored at −20° C.

Real-time PCR analysis of raphe slice cDNA for determination of relative levels of murine TPH mRNA was performed as follows. A 2.5 μL of cDNA was added to each well of a 96-well plate with 22.5 pL of TAQMAN reaction mix (1× Universal Master Mix (ABI); 20 nM forward and reverse 18S rRNA control primers with the 100 nM 18S rRNA control probe and either (a) 300 nM mTPH2-514F and mTPH2-585R primers with the 200 nM mTPH2-565T probe (TPH2-514 primer/probe set) or (b) 300 nM mTPH2-1270F and mTPH2-1344R primers with the 200 nM mTPH2-1292T probe (TPH2-1270 primer /probe set)). Samples were run on an ABI PRISM 7700 Sequence Detection Instrument (Applied Biosystems, Foster City, Calif.) and collected data was analyzed using Merck Biometrics TAQMANPLUS program.

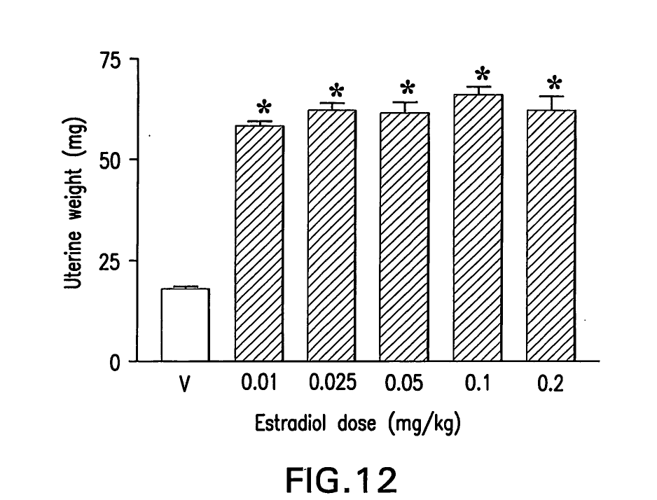

The results of Example 1 had suggested that while 17β-estradiol caused an induction of TPH1 mRNA expression in the DRN of ovariectomized mice as determined by in situ hybridization, it had no effect on TPH2 expression. The real-time PCR method confirmed the differential 17β-estradiol effect observed in Example 1. As shown in FIG. 4B, 17β-estradiol from 0.01 to 0.2 mpk, s.c., caused a significant induction in TPH1 mRNA levels of about 1.4 to 1.6 fold in the DRN of ovariectomized mice treated once daily with 17β-estradiol (p<0.01 for 0.025 to 0.1 mpk 17β-estradiol and p<0.001 for 0.2 mpk 17β-estradiol). In contrast, as shown in FIG. 4C, 17β-estradiol administered over the same concentration range had no effect on TPH2 mRNA levels. FIG. 12, shows that uterine weight measurements are dramatically induced by 17β-estradiol.

FIG. 6 shows that glucocorticoids did not affect TPH1 mRNA levels in the DRN of ovariectomized mice as measured by real-time PCR. The Figure shows that whereas 17β-estradiol at 0.2 mpk caused a significant 1.8 fold induction of TPH1 mRNA levels (p<0.001), neither 3 mpk of dexamethasone nor 10 mpk prednisolone had any effect on TPH1 mRNA levels.

In contrast, FIG. 7 shows that mifepristone (RU486) at 10 mpk, like dexamethasone at 3 mpk and prednisolone at 10 mpk, caused a decrease in TPH2 mRNA levels in the DRN of ovariectomized mice using either the TPH2-514 or the TPH2-1270 primer/probe set. In general, dexamethasone and prednisolone are full glucocorticoid receptor agonists whereas RU486 is a glucocorticoid receptor antagonist with some agonist activity (mixed or partial glucocorticoid receptor agonist). The result in FIG. 8 clearly shows the agonist activity of the RU486.

FIG. 8 shows that a further reduction of TPH2 mRNA levels was observed by increasing the concentration of RU486 from 10 mpk to 20 mpk. A similar further reduction was observed when the amount of dexamethasone was increased from 3 mpk to 10 mpk.

FIG. 9 shows that by co-administering the RU486 and dexamethasone, the glucocorticoid antagonist activity of the RU486 can be unmasked. As shown, dexamethasone or RU486 caused a significant decrease in TPH2 mRNA levels (40% and 28%, respectively). However, when the dexamethasone is administered with RU486, the TPH2 mRNA levels were reduced only by 17%. This effect on TPH2 mRNA levels is statistically different from the dexamethasone effect but not statistically different from the vehicle control value. The result indicates that the above method can be used to identify mixed or partial agonists. Thus, a full agonist in the presence of dexamethasone enhances the reduction of TPH2 mRNA expression by dexamethasone whereas a mixed or partial agonist would antagonize the reduction of TPH2 mRNA expression by dexamethasone. Thus, the method of the present invention can identify both analytes which affect TPH2 mRNA expression and analytes which are mixed or partial agonists.

### EXAMPLE 3

This example illustrates that the glucocorticoid effect on TPH2 mRNA expression observed in ovariectomized female mice is also observed in male mice undergoing chronic corticosterone treatment.

Male BKTO mice (Bantin and Kingman, Hull UK) weighing 25 g at the start of the experiment were briefly anaesthetized using isofluorane. Animals were implanted with 4×5 mg cortic/sterone pellets (Innovative Research of America, catalogue number G-111, 21 day release formulation) or 4× placebo pellets (catalogue number NC-111) this gave an equivalent of 40mg/kg/day of corticosterone. Pellets were implanted using a trochar supplied by the manufacturer which had been modified to allow 4 pellets to be held in one trochar, thus requiring only one incision per animal. The most ideal place to implant the pellet was where there is maximal space between the skin and the muscle such as the lateral side of the neck between the ear and the shoulder-no stitching or wound clipping was required when using this technique. Animals were returned to the home cage and allowed to recover. Fourteen days later, the animals were humanely killed and trunk blood collected into EDTA coated tubes, centrifuged for 10 minutes at 3000 rpm, and plasma removed and frozen on dry ice until ready for analysis. Brains were removed, frozen in isopentane, and stored at −80° C. until ready for analysis. Three to four 250 μm thick coronal sections of dorsal raphe were cut, the cortices were removed from the sections, and the sections were placed in RNALATER at 4° C. Dorsal raphe slices were processed for real-time RT-PCR analyses (TAQMAN) using the TPH2-514 primer/probe set and the PCR conditions in Example 2. Plasma corticosterone was analyzed using a COAT-A-COUNT rat corticosterone radioimmunoassay kit (DPC Products-Order no TKRC1). Analysis of samples was carried out as per manufacturer's instructions.

FIG. 10 shows a significant increase in cortisone levels in plasma of male mice after 14 days of cortisone treatment (p=0.004). FIG. 11 shows that in raphe slice preparations of the same mice, there was a significant decrease in TPH2 mRNA levels (p=0.01). These results show that the glucocorticoid effect on TPH2 expression is not limited to ovariectomized female mice. This indicates that the method of the present invention can be used to identify analytes which affect TPH2 mRNA levels in both male and female mice.

### EXAMPLE 4

This example shows that dexamethasone reduces TPH2 mRNA levels in the DRN of both ovariectomized female and intact male mice. The reduction of TPH2 mRNA in the DRN by dexamethasone is blocked by coadministration of mifepristone.

Ovariectomized C57/BL6 female mice (13 to 16 wks of age) were from Charles River Laboratories and intact C57/BL6 male mice (13 to 16 wks of age) were from Taconic Laboratories. Animals were fed a soy-free rodent chow and allowed a minimum of one week to recuperate from surgery and shipping. Mice were dosed subcutaneously in the morning (once daily for 4 days) with 0.1 cc of vehicle (sesame oil) or compound (dose is indicated for each experiment). Approximately six hours following the fourth dose, mice were deeply anesthetized with ketamine/xylazine.

For real-time RT-PCR analyses brains were removed from the skull, placed ventral side up in a mouse brain block on ice, and ice-cold razor blades are inserted into the block at 1 mm intervals. The caudal extent of the hypothalamus was used as an anatomical marker for the placement of the first razor blade, and 4 blades are placed in sequential slots, caudally. The four sections are examined and the two that encompass the greatest extent of the dorsal raphe are placed in a tube containing RNA-later and placed at 4° C. overnight. Brain slices were removed from RNALATER (Ambion) after 24 hr and stored at −80° C. in fresh tubes. An n of seven animals was used per treatment group for real-time RT-PCR studies. For in situ hybridization histochemistry brains are removed from the skull and placed ventral side up on powdered dry ice and frozen brains are stored at −80° C. until sectioning. An n of 5 to 6 animals was used per treatment group for in situ hybridization histochemistry analyses.

The murine TPH2 primers were mTPH2-514F and mTPH2-585R and the murine TPH2 probe was mTPH2-565T. mTPH2-565T was labeled with the fluorescent dye FAM and the quencher TAMRA.

Isolation of total RNA from murine raphe slices for TAQMAN analysis has been described in Example 2. Genomic DNA was removed from the total RNA samples using the DNA-FREE kit (Ambion) and cDNA synthesis performed using Multiscribe RT and the TaqMan Gold RT reagents (ABI). Reverse transcription was halted by the addition of EDTA. Samples were transferred to a storage plate and stored at −20° C.

Real-time PCR analyses using TAQMAN was as follows. A 2.5 μL of cDNA was added to each well of a 96well plate with 22.5 μl of TaqMan® reaction mix (1× Universal Master Mix (ABI), 20 nM forward and reverse 18S rRNA control primers, and 100 nM 18S rRNA control probe (ABI) and 300 nM mTPH2-514F and mTPH2-585R primers, 200 nM mTPH2-565T probe. Samples were run on an ABI PRISM 7700 Sequence Detection Instrument (Applied Biosystems (ABI), Foster City, Calif.) and collected data was analyzed using Merck Biometrics TAQMAN Plus program and statistical significance determined using a one-way ANOVA (CMG, Merck Biometrics).

In Situ Hybridization Histochemistry was as follows. Non-overlapping TPH2 riboprobe templates TPH2a and TPH2b were generated as described in Example 1. Both antisense and sense riboprobes were synthesized using ³³P or ³⁵S-labeled UTP incorporated into cRNA using the Promega Riboprobe System and the T7 polymerase for sense probes and the T3 polymerase for antisense probes.

Coronal sections (Cryoton, 16 mm in thickness) were hybridized with either murine TPH2 riboprobe TPH2a or TPH2b. Hybridized slides were exposed to Kodak Biomax MR film (Rochester, N.Y.) at room temperature for 3 to 16 hr. Autoradiographic images of midbrain sections were anatomically matched between animals, and densitometry performed using a CCD video camera (Dage-MTI Inc) fitted with Nikon lenses (Nikon Canada, Inc.), and the Scion Image Program The average gray scale optical density was obtained by subtracting the background reading outside the region of interest from the O.D. of the DRN. Analysis was performed on 5 to 6 coronal sections, representing the rostral to caudal extent of the DRN. The O.D. values from each animal were averaged, then the average of each group was calculated and reported as the mean±SEM. A One-way ANOVA compared treatment group averages (CMG, Merck Biometrics).

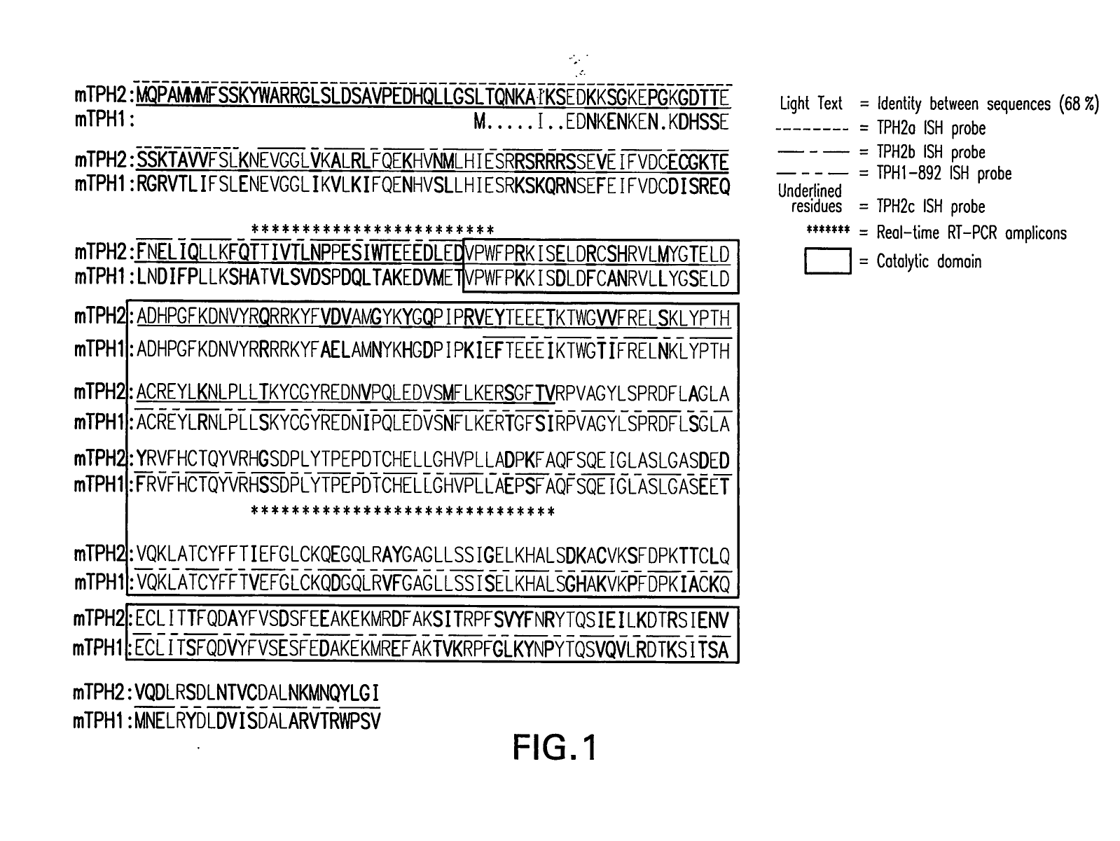

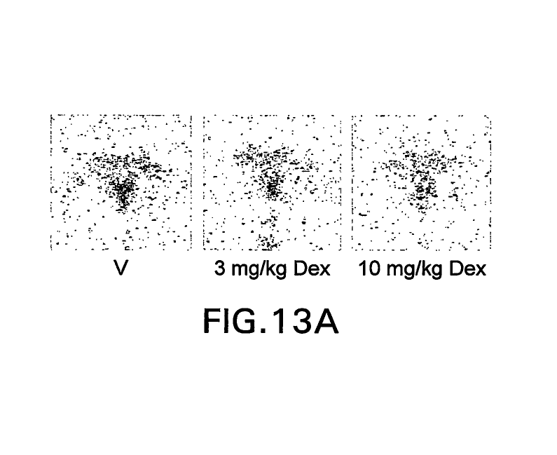

Treatment of ovariectomized female mice once daily for four days with dexamethasone reduces TPH2 mRNA levels by 28% at 3 mg/kg and 36% at 10 mg/kg as determined by densitometric analysis of autoradiographic images from in situ hybridization histochemical studies (FIGS. 13A and 1B; p<0.001, n=5 to 6).

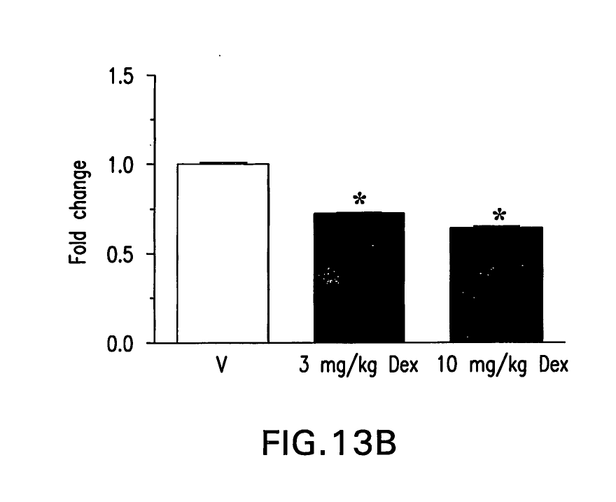

A real-time RT-PCR method was developed to quantify changes in TPH2 mRNA levels caused by glucocorticoids. One mm coronal sections of mouse brain were dissected at the level of Bregma 8.00 (See FIG. 4A) containing the DRN, cortices were removed and sections used for the isolation of total RNA and synthesis of cDNA for real-time RT-PCR analyses. Treatment of ovariectomized female mice once daily for four days with a range of dexamethasone doses from 0.1 to 3 mg/kg resulted in a reduction of TPH2 mRNA levels from 23% at 0.1 mg/kg to 44% at 3 mglkg in the raphe slice preparation (FIG. 14). These data confirmed the results observed with in situ hybridization histochemistry and show that at dexamethasone doses as low as 0.1 mg/kg, a statistically significant reduction in TPH2 mRNA levels was detected in the mouse DRN.

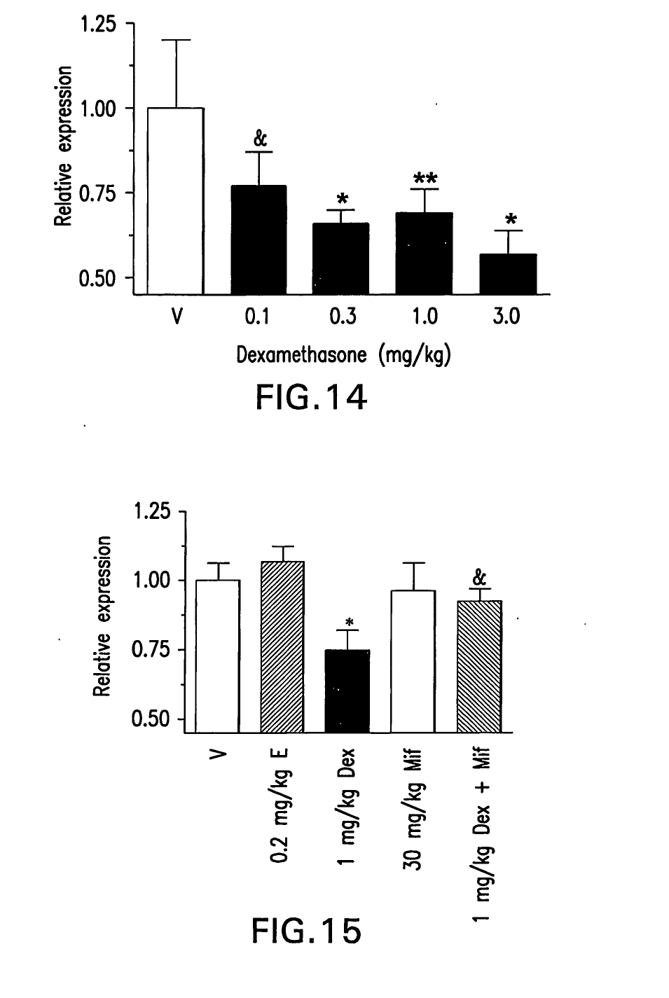

To test the gender specificity of glucocorticoid regulation of TPH2 message levels, 1 mpk dexamethasone was administered once daily for four days to intact male C57/B16 mice. The administration of dexamethasone resulted in a 26% reduction in TPH2 mRNA levels in the murine raphe slice preparation (FIG. 15) nearly equivalent to the 30% reduction in TPH2 message levels detected in ovariectomized female C57/B16 mice at 1 mg/kg as shown in FIG. 14. Treatment with mifepristone alone has no effect on TPH2 message levels. Similarly, treatment with 17β-stradiol has no effect on TPH2 mRNA levels.

Dexamethasone has high affinity for both the glucocorticoid and mineralocorticoid receptors; therefore, to examine the specificity of the glucocorticoid effect on TPH2 mRNA, dexamethasone was co-administered with mifepristone (RU486), a glucocorticoid receptor modulator with significant antagonist properties. As shown in FIG. 9, in ovariectomized female mice treated with 3 mg/kg dexamethasone, there was a 40% reduction in TPH2 mRNA levels, the reduction of which could be blocked by co-administration of mifepristone. Similarly, in intact male mice, administration of 1 mg/kg dexamethasone resulted in a 26% reduction in TPH2 mRNA levels. Like in the ovariectomized female mice, the reduction in mRNA levels could be blocked by co-administration of mifepristone (See FIG. 15). Partial agonist activity of mifepristone was detected in the overiectomized female mice at 20 mg/kg (28% reduction in TPH2 mRNA levels (See FIG. 9)) but not in the intact male mice when dosed at 30 mg/kg (FIG. 15). Mifepristone antagonism of dexamethasone mediated changes in TPH2 message levels indicated that dexamethasone was acting through the glucocorticoid receptor.

The results shown in Examples 1 to 4 demonstrate that TPH2 mRNA is differentially modulated by hormones in the murine dorsal raphe nucleus and that (1) glucocorticoid treatment results in a significant decrease in TPH2 mRNA levels following four days of daily dosing; (2) the glucocorticoid mediated reduction in TPH2 message levels is blocked by coadministration of the glucocorticoid receptor antagonist mifepristone; and (3) the effect of glucocorticoids on TPH2 message levels is not sex specific and was seen in both ovariectomized female and intact male C57/B16 mice.

In contrast, 17β-estradiol did not have a measureable effect on TPH2 message levels in the dorsal raphe nucleus following a four day dosing regimen in either ovariectomized female or intact male C57/B16 mice. 17β-estradiol has been shown to regulate TPH message and protein levels in the dorsal raphe of both macacque monkey and guinea pig (Bethea et al, Biol. PsychiaL 47: 562-576 (2000); Lu et al, Endocrine 11: 257-267 (1999); Pecins-Thompson et al, J. Neurosci. 16: 7021-7029 (1996)). Since changes in TPH2 mRNA in response to 17β-estradiol were not detected, it appears that the changes being measured in Bethea et al., Lu et al., and Pecins-Thompson et al., above correspond to changes in the expression of the TPH1 isoform.

There is much evidence in the literature describing the adrenalcortical influence and glucocorticoid modulation of TPH activity and protein in both neonatal and adult rat brain but there has been very little published describing the effects of glucocorticoids on TPH expression in the mouse midbrain or brainstem. Bilateral adrenalectomy has been shown to result in a decrease in TPH activity in adult rat midbrain that can be restored to baseline levels by administering corticosterone (Azmitia and McEwen, Science 166: 1274-1276 (1969); Rastogi and Singhal, J. Neural Trasm. 42: 63-71 (1978). In addition, it has been shown that stressors such as electric foot shock and cold exposure, which are known to cause increases in plasma corticosterone, result in increasing TPH activity in the adult rat midbrain (Azrnitia and McEwen, Brain Research 78: 291-302 (1974)). Bilateral adrenalectomy blocks the changes in TPH activity that are seen with stressors and administration of corticosterone restores the effect that stress has on TPH activity levels in adrenalectomized animals (Azmitia and McEwen, ibid). It has also been shown that changes in TPH activity correlate positively with changes in TPH protein (Azmitia et al. J. Neurosci. 13: 5041-5055 (1993). Indirect regulation of TPH enzyme activity has been achieved through icv injection of corticotrophin releasing factor (CRF), which results in an increase in TPH activity in adult rats that can be blocked by bilateral adrenalectomy and mifepristone and restored by administration of dexamethasone (Singh et al., Neurochem. Int. 20: 81-92 (1992). In these studies lesion of the central nucleus of the amygdala also blocked the effects of hypothalamic pituitary adrenal (HPA) axis activation on TPH activity suggesting that this brain region plays a particularly prominent role in adrenalcortical regulation of serotonin synthesis (Singh et al., ibid.).

There is, however, a dearth of information relating to glucocorticoid regulation of TPH activity or protein in the mouse brain. In one study, it was shown that TPH activity was unaffected by adrenalectomy or chronic corticosterone administration in adult mouse brain (Sze et al., J. Neurochem 26:169-173 (1976)). In contrast, adrenalectomy was shown to abolish the reserpine induced increase in TPH activity observed in adult mouse brainstem and corticosterone restored the reserpine induced increase in TPH activity in adrenalectomized mice (Sze et al., ibid.). Together the rodent studies clearly show that serotonin biosynthesis can be regulated by glucocorticoids and suggest that TPH activity may be relevant to the serotonergic tone of the limbic system. However, these studies do not identify the TPH isoform responsible for the changes in activity nor do they address the effects of adrenalcortical modulation on TPH activity in mouse brain.

Until recently, it was thought that a single TPH isoform was responsible for biosynthesis of serotonin. The existence of a second TPH isoform became evident when generation of mice no longer expressing TPH still exhibited serotonin biosynthesis in the midbrain and brainstem (Walther et al., Science 299: 76 (2003)). Silico screening of genomic databases resulted in the identification of a distinct gene with homology to the known TPH (Walther et al., ibid.). Consequently, human, mouse and rat clones for TPH2 were identified and shown to exhibit TPH activity when exogenously expressed in COS cells (Walther et al, ibid.). Walther et al. used RNA protection assays to show that TPH2 is highly expressed in the brain and from this data they conclude that TPH2 is the predominant TPH isoform in the brain and that TPH1 is the predominant TPH isoform expressed in the periphery. That TPH2 is the predominant TPH isoform in the rodent brain has been confirmed using in situ hybridization histochemistry to show that TPH2 mRNA is robustly expressed throughout the histologically defined raphe nuclei of the brainstem and midbrain as compared to TPH1 mRNA which is expressed at very low levels in the rodent dorsal raphe nucleus, but is robustly expressed in the pineal gland (herein; Patel et al., Biol. Psychiatry 55:428-33 (2004)). The identification of TPH2 helps clarify the apparent discrepancies that have existed in the literature between the reported weak TPH (TPH1) message levels in the raphe nuclei and the robust TPH immunoreactivity that was detected using antibodies made to epitopes shared by both TPH isoforms. The identification of TPH2 was a significant contribution because it is now possible to reexamine the regulation of TPH mRNA in the CNS and relate mRNA regulation to serotonin biosynthesis.

The Examples herein demonstrates that TPH2 mRNA is differentially regulated by hormones in the DRN. While dexamethasone treatment resulted in a significant decrease in TPH2 mRNA levels in both male and female mice, 17β-estradiol did not alter TPH2 message levels in the DRN. TPH1 mRNA also appears to be differentially regulated by hormones in the DRN of both male and female mice. 17β-estradiol induces TPH1 message levels in the dorsal raphe nucleus (data not shown). Since estradiol has no effect on TPH1 message levels in ER-βknockout mice, it appears that estradiol-mediated changes in TPH1 message are mediated through activation of ER-β. In contrast, dexamethasone has no detectable effect of TPH1 mRNA in the DRN of male or female mice (data not shown). The results herein suggest that estrogen and glucocorticoids may differentially regulate the murine serotonergic system at the level of 5-HT biosynthesis.

Serotonin neurotransmission plays a major role in behavioral, emotional and cognitive processes and because of this there has been intense interest in the field of psychiatry to study polymorphisms in TPH. This intense interest in TPH polymorphisms has led to the recent publication of data from several SNP analyses that suggest the existence of an affective disorder-associated haplotype in the TPH2 gene (Zill et al., Molec. Psychiatry 1-7, (advance online publication 4 May 2004); Harvey et al., advance online publication (20 Jul. 2004); Luca et al., advance online publication (15 Jun. 2004)). This genetic data suggests that changes in expression and/or function of TPH2 may be associated with psychiatric disease and emphasizes the need to understand what makes TPH isoforms distinct with regard to function and regulation. Glucocorticoid-mediated reduction of TPH2 message may have relevance to the etiology of major depression, psychotic major depression in particular, where elevated glucocorticoids are one hallmark of the disease. (Belanoff et al., Am. J. Psychiatry 158:1612-1616 (2001))

### EXAMPLE 5

This example describes a method for making polyclonal antibodies specific for the TPH2.

A peptide corresponding to an epitope of the TPH2 with an amino acid sequence with little identity to any amino acid sequence in TPH1 (for example a peptide corresponding to an amino acid sequence within a region encompassed by the TPH2a or TPH2b probes. Antibodies are generated in New Zealand white rabbits over a 10-week period. The peptide is preferably conjugated to a protein such as keyhole limpet hemocyanin (KLH), emulsified by mixing with an equal volume of Freund's complete adjuvant and injected into three subcutaneous dorsal sites for a total of 0.1 mg peptide per immunization. A booster containing about 0.1 mg peptide emulsified in an equal volume of Freund's incomplete adjuvant is administered subcutaneously two weeks later. Animals are bled from the articular artery. The blood is allowed to clot and the serum collected by centrifugation. The serum is stored at −20° C.

For purification, TPH2 or the peptide is immobilized on an activated support. Antisera is passed through the sera column and then washed. Specific antibodies are eluted via a pH gradient, collected, and stored in a borate buffer (0.125M total borate) at −0.25 mg/mL. The anti-TPH2 antibody titers are determined using ELISA methodology with free TPH2 or peptide bound in solid phase (1 pg/well). Detection is obtained using biotinylated anti-rabbit IgG, HRP-SA conjugate, and ABTS.

### EXAMPLE 6

This example describes a method for making monoclonal antibodies specific for the TPH2.

BALB/c mice are immunized with an initial injection of about 1 μg of purified peptide as in Example 4 per mouse mixed 1:1 with Freund's complete adjuvant. After two weeks, a booster injection of about 1 μg of the antigen is injected into each mouse intravenously without adjuvant. Three days after the booster injection serum from each of the mice is checked for antibodies specific for the TPH2.

The spleens are removed from mice positive for antibodies specific for cDkk-4 protein and washed three times with serum-free DMEM and placed in a sterile Petri dish containing about 20 mL of DMEM containing 20% fetal bovine serum, 1 mM pyruvate, 100 units penicillin, and 100 units streptomycin. The cells are released by perfusion with a 23 gauge needle. Afterwards, the cells are pelleted by low-speed centrifugation and the cell pellet is resuspended in 5 mL 0.17 M ammonium chloride and placed on ice for several minutes. Then 5 mL of 20% bovine fetal serum is added and the cells pelleted by low-speed centrifugation. The cells are then resuspended in 10 mL DM and mixed with mid-log phase myeloma cells in serum-free DMEM to give a ratio of 3:1. The cell mixture is pelleted by low-speed centrifugation, the supernatant fraction removed, and the pellet allowed to stand for 5 minutes. Next, over a period of 1 minute, 1 mL of 50% polyethylene glycol (PEG) in 0.01 M HEPES, pH 8.1, at 37° C. is added. After 1 minute incubation at 37° C., 1 mL of DMEM is added for a period of another 1 minute, then a third addition of DMEM is added for a further period of 1 minute. Finally, 10 mL of DMEM is added over a period of 2 minutes. Afterwards, the cells are pelleted by low-speed centrifugation and the pellet resuspended in DMEM containing 20% fetal bovine serum, 0.016 mM thymidine, 0.1 hypoxanthine, 0.5 μM aminopterin, and 10% hybridoma cloning factor (HAT medium). The cells are then plated into 96-well plates.

After 3, 5, and 7 days, half the medium in the plates is removed and replaced with fresh HAT medium. After 11 days, the hybridoma cell supernatant is screened by an ELISA assay. In this assay, 96-well plates are coated with TPH2 or the peptide. One hundred μL of supernatant from each well is added to a corresponding well on a screening plate and incubated for 1 hour at room temperature. After incubation, each well is washed three times with water and 100 μL of a horseradish peroxide conjugate of goat anti-mouse IgG (H+L), A, M (1:1,500 dilution) is added to each well and incubated for 1 hour at room temperature. Afterwards, the wells are washed three times with water and the substrate OPD/hydrogen peroxide is added and the reaction is allowed to proceed for about 15 minutes at room temperature. Then 100 μL of 1 M HCl is added to stop the reaction and the absorbance of the wells is measured at 490 nm. Cultures that have an absorbance greater than the control wells are removed to two cm² culture dishes, with the addition of normal mouse spleen cells in HAT medium. After a further three days, the cultures are re-screened as above and those that are positive are cloned by limiting dilution. The cells in each two cm² culture dish are counted and the cell concentration adjusted to 1×10⁵ cells per mL. The cells are diluted in complete medium and normal mouse spleen cells are added. The cells are plated in 96-well plates for each dilution. After 10 days, the cells are screened for growth. The growth positive wells are screened for antibody production; those testing positive are expanded to 2 cm² cultures and provided with normal mouse spleen cells. This cloning procedure is repeated until stable antibody producing hybridomas are obtained. The stable hybridomas are progressively expanded to larger culture dishes to provide stocks of the cells.

Production of ascites fluid is performed by injecting intraperitoneally 0.5 mL of pristane into female mice to prime the mice for ascites production. After 10 to 60 days, 4.5×10⁶ cells are injected intraperitoneally into each mouse and ascites fluid is harvested between 7 and 14 days later.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method for identifying an analyte which directly or indirectly modulates activity of a glucocorticoid receptor in the brain of an animal, which comprises:
(a) administering a glucocorticoid to a first animal and measuring an amount of tryptophan hydroxylase (TPH2) in the brain of the first animal; and
(b) administering the glucocorticoid and the analyte to a second animal and measuring the amount of the TPH2 in the brain of the second animal wherein a change in the amount of the TPH2 in the brain of the second animal relative to the amount of the TPH2 in the first animal indicates that the analyte modulates the activity of the glucocorticoid receptor in the brain of the animal.

2. The method of claim 1 wherein the TPH2 is TPH2 mRNA and the TPH2 mRNA is measured by reverse transcription-polymerase chain reaction (RT-PCR).

3. The method of claim 2 wherein the RT-PCR is a real-time RT-PCR which uses an oligonucleotide probe comprising a nucleotide sequence complementary to a nucleotide sequence comprising the TPH2 mRNA.

4. The method of claim 1 wherein the TPH2 is TPH2 RNA and the TPH2 mRNA is measured by in situ hybridization which uses an oligonucleotide probe comprising a nucleotide sequence complementary to a nucleotide sequence comprising the TPH2 mRNA.

5-8. (canceled)

9. A method for determining whether an analyte directly or indirectly affects the amount of tryptophan hydroxylase isoform 2 (TPH2) in the brain of an animal which has chronically elevated glucocorticoid levels, which comprises:
(a) providing an animal which has the chronically elevated glucocorticoid levels;
(b) administering the analyte to the animal which has the chronically elevated glucocorticoid levels; and
(c) measuring the amount of the TPH2 in the brain of the animal wherein an increase in the amount of the TPH2 in the brain of the animal compared to the amount of the TPH2 in the brain of the animal without the analyte indicates that the analyte has an effect on the amount of the TPH2 in the animal which has the chronically elevated glucocorticoid levels.

10. The method of claim 9 wherein the TPH2 is TPH2 mRNA and the TPH2 mRNA is measured by reverse transcription-polymerase chain reaction (RT-PCR).

11. The method of claim 10 wherein the RT-PCR is a real-time RT-PCR which uses an oligonucleotide probe comprising a nucleotide sequence complementary to a nucleotide sequence comprising the TPH2 mRNA.

12. The method of claim 9 wherein the TPH2 is TPH2 RNA and the TPH2 mRNA is measured by in situ hybridization which uses an oligonucleotide probe comprising a nucleotide sequence complementary to a nucleotide sequence comprising the TPH2 mRNA.

13-16. (canceled)

17. A method for determining whether an analyte is a full glucocorticoid agonist or antagonist or partial glucocorticoid agonist in the brain of an animal, which comprises:
(a) administering the analyte to a first animal and measuring an amount of tryptophan hydroxylase isoform 2 (TPH2) in the brain of the first animal;
(b) administering a glucocorticoid to a second animal and measuring an amount of the TPH2 in the brain of the second animal;
(c) administering the glucocorticoid and the analyte to a third animal and measuring an amount of the TPH2 in the brain of the third animal; and
(d) comparing the amount of the TPH2 in the brains of the first, second, and third animals wherein (1) a decrease in the TPH2 in the brain of the first animal and a decrease in the TPH2 in the brain of the second animal which is not greater than the decrease in the TPH2 in the brain of the third animal indicates that the analyte is a full agonist, (2) an increase in TPH2 in the brain of the first animal and an increase in the TPH2 in the brain of the third animal compared to the TPH2 in the brain of the second animal indicates that the analyte is a full antagonist, and (3) a decrease in the TPH2 in the brain of the first animal and a decrease in the TPH2 in the brain of the second animal which is greater than the decrease in the TPH2 in the brain of the third animal indicates that the analyte is a partial agonist.

18. The method of claim 17 wherein the TPH2 is TPH2 mRNA and the TPH2 mRNA is measured by reverse transcription-polymerase chain reaction (RT-PCR).

19. The method of claim 18 wherein the RT-PCR is a real-time RT-PCR which uses an oligonucleotide probe comprising a nucleotide sequence complementary to a nucleotide sequence comprising the TPH2 mRNA.

20. The method of claim 17 wherein the TPH2 is TPH2 RNA and the TPH2 mRNA is measured by in situ hybridization which uses an oligonucleotide probe comprising a nucleotide sequence complementary to a nucleotide sequence comprising the TPH2 mRNA.

21-28. (canceled)
